(19) United States
(12) Patent Application Publication
(10) Pub. No.: US 2007/0148762 A1
(43) Pub. Date: Jun. 28, 2007

(54) Cell culture patterning substrate

(76) Inventors: Hideyuki Miyake, Tokyo (JP); Hideshi Hattori, Tokyo (JP); Hironori Kobayashi, Tokyo (JP); Hidetsugu Tazawa, Tokyo (JP)

Correspondence Address: LADAS & PARRY LLP, 224 SOUTH MICHIGAN AVENUE, SUITE 1600, CHICAGO, IL, 60604, US

(21) Appl. No.: 10/589,376

(22) PCT Filed: Feb. 18, 2005
(86) PCT No.: PCT/JP05/02608

§ 371 Date: Aug. 14, 2006

(30) Foreign Application Priority Data
Feb. 19, 2004 (JP) 2004-043367

(51) Int. Cl. C12M 3/00

(52) U.S. Cl. 435/289.1

(57) ABSTRACT

A cell culture patterning substrate on which cells can be arranged regularly with high efficiency over a large area on a base material so as to attain formation of a tissue, etc. The cell culture patterning substrate includes: a base material; and a cell culture region which is formed on the base material, is a region for culturing a cell and contains a cell adhesive layer having adhesive properties to the cell. The cell culture region includes: a cell adhesion portion at which the cell adhesive layer is formed; and a cell adhesion auxiliary portion, formed in a pattern, which inhibits adhesion to the cell, and the cell adhesion auxiliary portion is formed such that, upon adhesion of the cell to the cell adhesion portion, the cells on two cell adhesion portions adjacent to the cell adhesion auxiliary portion can be bound to each other on the cell adhesion auxiliary portion.

### TECHNICAL FIELD

The present invention relates to a cell culture patterning substrate used, for example, in culturing cells such as in blood vessels etc.

### BACKGROUND ART

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method for arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in the patent document 1, an electric charge-retaining medium on which an electrostatic pattern is formed is applied to culture cells for the purpose of proliferating nerve cells in a form of circuit, and the like. Furthermore, the patent document 2 tries to arrange cultured cells on a surface on which a cell adhesion-inhibiting or cell adhesive photosensitive hydrophilic polymer has been patterned by a photolithography method.

Furthermore, the patent document 3 discloses a cell culture base material on which a substance such as collagen and the like affecting on the adhesion ratio and form of cells is patterned, and a method for producing this base material by a photolithography method. By culturing cells on such a base material, a larger amount of cells can be adhered on a surface on which collagen or the like is patterned, to realize patterning of cells.

However, in such cell culture methods, when the area of the cell culture pattern is large, cells can be arranged regularly in the edge part of the cell culture pattern. However, in the center part of the cell culture pattern, there is a problem that the cells may be poorly arranged or the cells may not be adhered. Moreover, ordinary cells form a tissue through a morphological change of individual cells. In Non-Patent Document 1 etc., it is described that, when cells are cultured in the above-mentioned cell culture pattern or the like in order to form such a tissue, the cells are stimulated by a boundary of a cell adhesion portion having adhesive properties to cells and a cell adhesion-inhibiting portion which inhibits adhesion to cells, thus generating a morphological change of the cells, and this morphological change gradually propagates toward the center part of the cell culture pattern. However, there is a problem that when the area of this cell culture pattern is large, the morphological change of the cells hardly propagates to the center part, so the cells in the center part are hardly morphologically changed, thus failing to form a tissue in the center part. There is another problem that cells are disseminated and adhered to a substrate, the adhesion of the cells is time-consuming.

Patent Document 1: Japanese Patent Application Laid-Open (JP-A) No. 2-245181

Patent Document 2: JP-A No. 3-7576

Patent Document 3: JP-A No. 5-176753

Non-Patent Document 1: Spargo et al., Proceedings of the National Academy of Sciences of the United States of America (1994) p. 11070-

### DISCLOSURE OF INVENTION

### Problem to be Solved by the Invention

Accordingly, it has been desired to provide a cell culture patterning substrate on which cells can be arranged regularly with high efficiency over a large area on a base material so as to attain formation of a tissue, etc.

### MEANS FOR SOLVING THE PROBLEM

The present invention provides a cell culture patterning substrate comprising: a base material; and a cell culture region which is formed on the base material, is a region for culturing a cell and contains a cell adhesive layer having adhesive properties to the cell, wherein the cell culture region comprises: a cell adhesion portion at which the cell adhesive layer is formed; and a cell adhesion auxiliary portion, formed in a pattern, which inhibits adhesion to the cell, and the cell adhesion auxiliary portion is formed such that, upon adhesion of the cell to the cell adhesion portion, the cells on two cell adhesion portions adjacent to the cell adhesion auxiliary portion can be bound to each other on the cell adhesion auxiliary portion.

In the present invention, since the cell adhesion auxiliary portion is formed in the cell culture region, when cells are adhered onto the cell adhesion portion, these cells can be activated so that the cells can be cultured efficiently in a short time. Further, since the cells are cultured per each region that are in between the cell adhesion auxiliary portions, the number of cells stimulated by the boundary regions can be made larger, compared to a case wherein the cells are cultured in the whole area of the cell culture region, with no cell adhesion auxiliary portions. Thereby, the cells can be in excellent arrangement, and also, morphologically change of the cells can be carried out uniformly. In the present invention, the cell adhesion auxiliary portion is formed such that, upon adhesion of cells to the cell adhesive layer, bonding of the cells adhered to the adjacent cell adhesion portions to each other are not prevented. Therefore, the cells on entire cell culture region can be finally bounded so that obtained tissue and the like can be made larger in area.

In the above-mentioned invention, the cell adhesion auxiliary portion may be formed in a line form in the cell culture region. In this case, there is an advantage that a design for forming the cell culture region becomes easy, and the cells are easily regularly arranged upon adhesion.

Further, in the above-mentioned invention, a boundary between the cell adhesion auxiliary portion and the cell adhesion portion may be formed in a pattern with concavoconvex. When the cells are adhered along the pattern with concavoconvex, the cells can receive more stimulation from the boundary regions so that the cells can be further regularly arranged. The adhesion of the cells to the cell adhesion portion can be activated so that the cells can be adhered onto the substrate efficiently in a short time with the cell culture patterning substrate.

Moreover, the present invention provides a cell culture patterning substrate comprising: a base material; and a cell culture region which is formed on the base material, is a region for culturing a cell and contains a cell adhesive layer having adhesive properties to the cell, wherein an edge part of the cell adhesive layer is formed in a pattern with concavoconvex.

In the present invention, since the edge part of the cell adhesive layer is formed in a pattern with concavoconvex, the cells can receive more stimulation from the boundary regions upon adhesion of the cells to the cell adhesive layer, thus realizing more lined up arrangement of the cells along the edge part of the cell adhesive layer. The adhesion of the cells to the cell adhesion portion can be activated so that the cells can be adhered onto the substrate efficiently in a short time with the cell culture patterning substrate.

In the above-mentioned invention, it is preferred that the distance between an edge part of the concave portion and an edge part of the convex portion of the concavoconvex, upon adhesion of the cell to the cell adhesive layer, is a size that the cells are aligned linearly. This is because such size of the concavoconvex realizes excellent arrangement of the cells.

In the above-mentioned invention, it is preferred that the average distance, between the edge part of the concave portion and the edge part of the convex portion of the concavoconvex, is in the range of 0.5 μm to 30 μm. This is because when the concavoconvex are in such a range, the cells can be excellently arranged and the cells can be activated.

### EFFECT OF THE INVENTION

According to the present invention, there is provided a cell culture patterning substrate wherein, upon the adhesion of the cells onto the cell adhesion portion, the cells can be activated so that the cells can be cultured efficiently in a short time over a large area. There is another effect that the cells can be made excellent in arrangement and the morphologically change of the cells can be carried out uniformly.

### BRIEF DESCRIPTION OF DRAWINGS

FIG. 1 is a schematic sectional view showing an example of the cell culture patterning substrate of the present invention.

FIG. 2 is a schematic sectional view showing another example of the cell culture patterning substrate of the present invention.

FIG. 3 is a schematic sectional view showing another example of the cell culture patterning substrate of the present invention.

FIG. 4 is a schematic sectional view showing another example of the cell culture patterning substrate of the present invention.

FIG. 5 is a schematic sectional view showing another example of the cell culture patterning substrate of the present invention.

FIG. 6 is a process chart showing an example of a method for forming cell adhesion auxiliary portion in the cell culture patterning substrate of the present invention.

FIG. 7 is a schematic sectional view showing an example of the photocatalyst-containing layer side substrate used in the present invention.

FIG. 8 is a schematic sectional view showing an example of the photocatalyst-containing layer side substrate used in the present invention.

FIG. 9 is a schematic sectional view showing an example of the photocatalyst-containing layer side substrate used in the present invention.

FIG. 10 is a process chart showing another example of a method for forming a cell adhesion auxiliary portion in the cell culture patterning substrate of the present invention.

FIG. 11 is a process chart showing an example of a method for forming a cell adhesive layer in the cell culture patterning substrate of the present invention.

FIG. 12 is a schematic sectional view showing another example of the cell culture patterning substrate of the present invention.

### DESCRIPTION OF SYMBOLS

1: Base material
2: Cell culture region
3: Cell adhesion portion
4: Cell adhesion auxiliary portion
5: Photomask
6: Energy

### BEST MODE FOR CARRYING OUT THE INVENTION

The present invention relates to a cell culture patterning substrate used in culturing cells, and there are two embodiments for the cell culture patterning substrate of the present invention. Hereinafter, the respective embodiments will be explained below separately.

### A. First Embodiment

Firstly, a first embodiment of the cell culture patterning substrate of the present invention will be explained. The first embodiment of the cell culture patterning substrate of the present invention is a cell culture patterning substrate comprising: a base material; and a cell culture region which is formed on the base material, is a region for culturing a cell and contains a cell adhesive layer having adhesive properties to the cell,
wherein the cell culture region comprises: a cell adhesion portion at which the cell adhesive layer is formed; and a cell adhesion auxiliary portion, formed in a pattern, which inhibits adhesion to the cell, and
the cell adhesion auxiliary portion is formed such that, upon adhesion of the cell to the cell adhesion portion, the cells on two cell adhesion portions adjacent to the cell adhesion auxiliary portion can be bound to each other on the cell adhesion auxiliary portion.

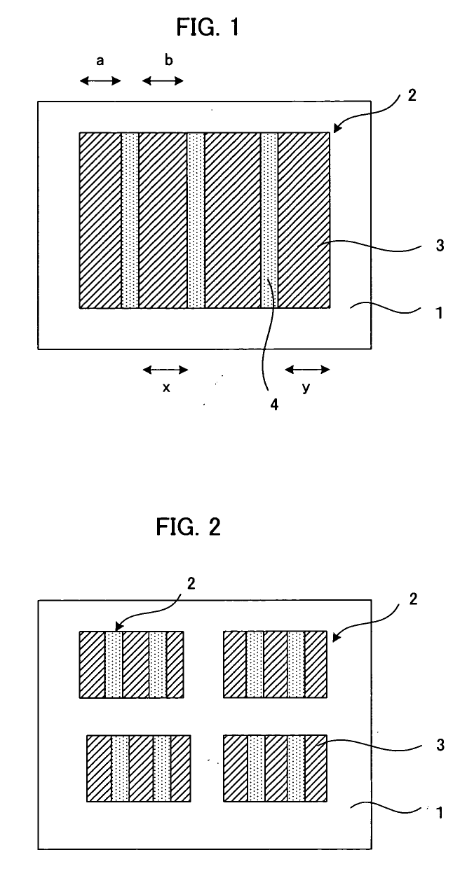

The cell culture patterning substrate of this embodiment is, for example as shown in FIG. 1, comprises a base material 1 and a cell culture region 2 formed on the base material 1, wherein the cell culture region 2 comprises a cell adhesion portion 3 having cell adhesive properties, with a cell adhesive layer formed thereon, and a cell adhesion auxiliary portion 4 inhibiting adhesion to cells.

Generally, when cells are adhered to a cell culture region and cultured to form a tissue, the cells are gradually arranged from the outside toward inside of the cell culture region. For forming a tissue, individual cells should be changed morphologically and arranged, and this morphological change also gradually occurs from the edge part toward center part of the cell culture region.

Accordingly, when an ordinary cell culture patterning substrate is used to culture the cells, in a case of a cell culture region for culturing cells of a large area, a tissue may not be formed in the center part because of insufficient arrangement of the cells, or the cells may fail to adhere to the center part of the cell culture portion. There is also a problem that the objective tissue is not formed because of the poor ability of cells to change morphologically in the center part.

According to the present invention, on the other hand, the cell adhesion auxiliary portion is formed in the cell culture region, and as shown in FIG. 1, for example, the cells are cultured in the cell adhesion portions 3 sandwiched between the cell adhesion auxiliary portions 4. That is, the arrangement and the morphological change of the cells can be generated starting from the boundary between the cell adhesion auxiliary portion 4 and the cell adhesion portion 3, and unlike a substrate not provided with the cell adhesion auxiliary portion 4, the substrate of the present invention can also be provided with a boundary region also inside of the cell culture region 2. Accordingly, the cells that have adhered to the cell culture region 2 can receive stimulation from the boundary between the cell adhesion portion 3 and the cell adhesion auxiliary portion 4 present inside of the cell culture region 2. The cells can thereby be made excellent in their ability to be arranged and morphologically changed in the whole area of the cell culture region 2.

In this embodiment, the cell adhesion auxiliary portion is formed so that the cells adhered onto the two adjacent cell adhesive layers can be bound to each other on the cell adhesion auxiliary portion. For example, as shown in FIG. 1, the cell adhesion auxiliary portion 4 is formed such that cells adhered onto the region “a” of the cell adhesion portion 3 and cells adhered onto the region “b” of the cell adhesion portion 3 can be bound to each other on the cell adhesion auxiliary portion 4. The cells can thereby be finally cultured in the same area as when the cells are cultured in the whole area of the cell culture region 2. This is because even if a region inhibiting adhesion to cells is present, when the cells are present on both sides of the region and close to each other so as to be influenced by each other, the cells can interact with each other also on the region inhibiting adhesion to the cells.

It is known that when the cell adhesive layer has defects etc., the cells are activated and are easily adhered to that region. In this embodiment, the cell adhesion auxiliary portion formed inside of the cell culture region can exhibit the same effect as these defects and the cells are activated. Thus, the cells can be adhered to the substrate efficiently in a short time.

Hereinafter, the respective components of the cell culture patterning substrate in this embodiment will be described in detail.

1. Cell Culture Region

First, the cell culture region in the cell culture patterning substrate in this embodiment is described. The cell culture region in this embodiment is a region formed for culturing cells, which is a region comprising: a cell adhesion portion with a cell adhesive layer having cell adhesive properties formed thereon; and a cell adhesion auxiliary portion formed in a pattern and inhibiting adhesion to cells.

In this embodiment, as shown in FIG. 1 for example, the cell culture region may be formed on a part of the base material 1 or the whole surface of the base material may be the cell culture region. For example, when the cell culture region 2 is formed on a part of the base material 1 as shown in FIG. 1 for example, the region other than the cell culture region on the base material 1 is a non-cell culture region which inhibits adhesion to cells. In this embodiment, the number of cell culture regions formed on one base material is not limited to one, and as shown in FIG. 2 for example, a plurality of cell culture regions 2 may be formed on the base material 1. In this case too, the regions other the cell culture regions on the base material 1 are the non-cell culture regions.

The size of each cell culture region, though varying depending on the size and type of the objective tissue, shall be usually in the range of 0.05 mm² to 8000 mm², particularly 0.1 mm² to 10 mm².

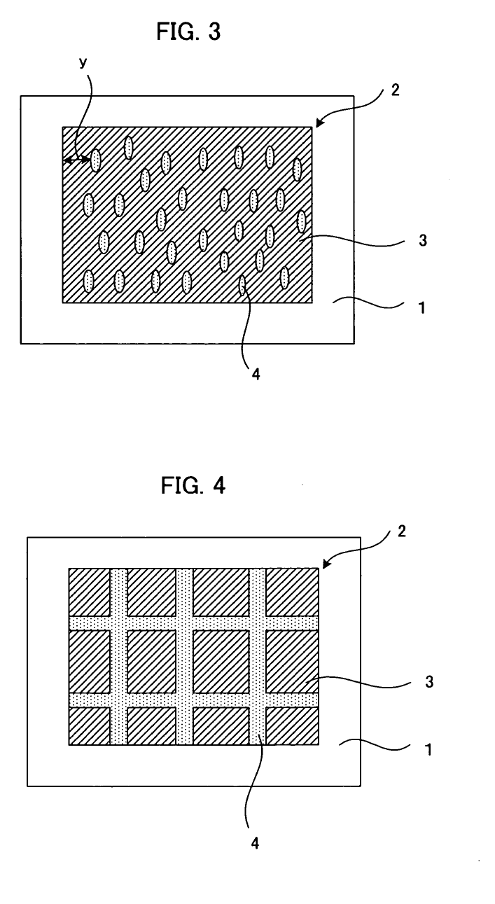

In the cell culture region described above, the cell adhesion auxiliary portion is formed in a pattern in the cell adhesion portion. In this embodiment, this cell adhesion auxiliary portion is not particularly limited insofar as the cell adhesion auxiliary portion is formed such that cells adhered to two cell adhesion portions adjacent to the cell adhesion auxiliary portion can be bound to each other on the cell adhesion auxiliary portion, and simultaneously the cells adhered to the cell adhesive layer are regularly arranged and the morphological change of the cells occurs uniformly. As shown in FIG. 1, for example, the cell adhesion auxiliary portions 4 may be formed in a line form in the cell culture region 2, or as shown in FIG. 3, for example, the cell adhesion auxiliary regions 4 may be formed randomly in the cell culture region 2.

The width of the cell adhesion auxiliary portion varies depending on the type and size of the cell cultured, and is usually in the range of preferably 0.5 μm to 10 μm, more preferably 1 μm to 5 μm. When the width is broader than the above range, the cells adhered to the two cell adhesion portions adjacent to the cell adhesion auxiliary portion hardly interact with each other on the cell adhesion auxiliary portion. While when the width is narrower than the above range, a pattern of such size is hardly accurately obtained by patterning techniques described later, and the cell adhesion auxiliary portion hardly exerts influence on the ability of cells to be arranged and morphologically changed as described above.

In this case, the width of the cell adhesion portion sandwiched between the cell adhesion auxiliary portions (for example, the distance represented by “x” in FIG. 1) or the width of the cell adhesion portion sandwiched between the cell adhesion auxiliary portion and the non-cell culture region (for example, the distance represented by “y” in FIG. 1) is selected suitably depending on the size and type of the cell cultured, the type of the objective tissue or the like, but is usually preferably in the range of 1 μm to 200 μm, particularly 40 μm to 80 μm. The cells adhered to the cell adhesion portion can thereby be regularly arranged and excellently morphologically changed to form a tissue.

In this embodiment, the cell adhesion auxiliary portion is particularly preferably formed in a line form. A design for forming the cell culture region can thereby become easy, and the cultured cells can have excellent arrangement ability. The “line form” means that the cell adhesion auxiliary portions are linearly formed. This includes not only a case wherein the cell adhesion auxiliary portion is formed continuously as shown in e.g. FIG. 1, but a case wherein the cell adhesion auxiliary portions are formed in broken lines. Also, in this embodiment, the cell adhesion auxiliary portion may be arranged in a line form in one direction, or as shown in e.g. FIG. 4, the cell adhesion auxiliary portion 4 may be arranged in a line form in a plurality of directions.

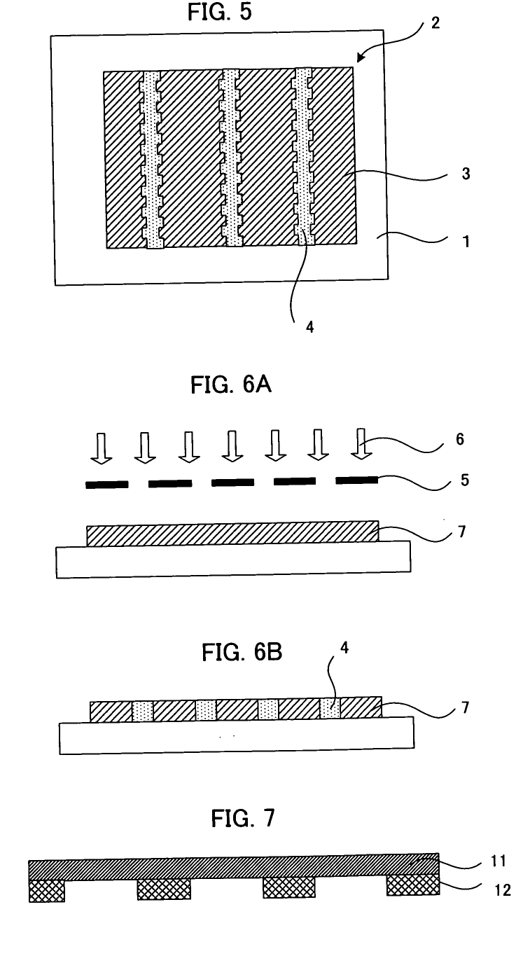

Further inn this embodiment, the boundary between the cell adhesion portion and the cell adhesion auxiliary portion may be arranged in a pattern with concavoconvex. By arranging cells along the pattern with concavoconvex, the cells can be arranged more regularly. In this case, there is also an advantage that the cells adhered thereto can be more activated so as the cells can be cultured efficiently. The pattern with concavoconvex is not particularly limited insofar as it is a pattern along which the cells can be regularly arranged, and for example, the boundary between the cell adhesion portion 3 and the cell adhesion auxiliary portion 4 may have concavoconvex having a right-angled shape as shown in FIG. 5 or may have concavoconvex having a wavy shape. Even if the cell adhesion auxiliary portion is formed for example in a wavy form or the cell adhesion auxiliary portion is formed in a randomly pattern, the boundary between the cell adhesion auxiliary portion and the cell adhesion portion can be formed in a pattern with concavoconvex. In this case too, the same effect can be obtained.

The distance between an edge part of the concave portion and an edge part of the convex portion of the concavoconvex is preferably such a size as to allow cells to be arranged linearly upon adhesion of the cells onto the cell adhesive layer. Specifically, the size is selected suitably depending on the shape etc. of the cells to be cultured, and usually the average distance between an edge part of the concave portion and an edge part of the convex portion of the concavoconvex is preferably in the range of 0.5 μm to 30 μm, particularly 1 μm to 5 μm. When the cells are cultured, the cells can thereby be cultured into an objective form to form a tissue, without cell deficiency at the edge part of the cell culture region. The average distance between the edge part of the concave portion and the edge part of the convex portion on the pattern with concavoconvex is a value determined by measuring the distances between the lowermost bottom and the uppermost top of each concavoconvex, within the range of 200 μm of the boundary between the cell adhesion portion and the cell adhesion auxiliary portion, and calculating the average thereof.

Hereinafter, the cell adhesion portion and the cell adhesion auxiliary portion, constituting the cell culture region, are respectively described in detail.

(Cell Adhesion Portion)

First, the cell adhesion portion used in this embodiment is described in detail. The cell adhesion portion in this embodiment is a region wherein a cell adhesive layer having cell adhesive properties is formed in a cell culture region on a base material. The cell adhesive layer is not particularly limited insofar as it has cell adhesive properties, and a layer having cell adhesive properties, used in a general cell culture patterning substrate, can be used. In this embodiment, a cell adhesion portion can be formed by forming the cell adhesive layer in a pattern. For example, the cell adhesion portion can be formed by applying, in a pattern, a cell adhesive layer forming coating solution containing a material having cell adhesive properties. Alternatively, the cell adhesive layer forming coating solution may be formed on the whole area of the cell culture region and the cell adhesion portion may be formed by photolithographic techniques etc.

In this embodiment, the cell adhesive layer is a layer containing a cell adhesive material. The cell adhesive material has cell adhesive properties and are decomposed or denatured by the action of a photocatalyst upon irradiation with energy. By irradiating this cell adhesive layer with energy, a patterning can be carried out to form the cell adhesion portion. In this case, the cell adhesive layer is formed for example on the whole surface of the cell culture region and then irradiated with energy, in a pattern of which the cell adhesion auxiliary portion will be formed, thereby decomposing or denaturing the cell adhesive material by the action of a photocatalyst to form a cell adhesion auxiliary portion, which inhibits adhesion to cells, and a cell adhesion portion having cell adhesive properties. The cell adhesive layer containing such cell adhesive material, the method for forming the cell adhesion auxiliary portion, etc. will be described later in more detail.

The cell adhesive layer used in this embodiment may be formed so as the cell adhesion-inhibiting material is decomposed or denatured to obtain cell adhesive properties by: coating a cell adhesion-inhibiting layer, onto the whole surface of the cell culture region, containing a cell adhesion-inhibiting material having cell adhesion-inhibiting properties and are decomposed by the action of a photocatalyst upon irradiation with energy; and then irradiating, with energy, a region other than the cell adhesion auxiliary portion. In this case, since the region other than the region irradiated with energy to form the cell adhesion portion is a region which inhibits adhesion to cells, the region can be used as the cell adhesion auxiliary portion. The cell adhesion-inhibiting layer containing the cell adhesion-inhibiting material, the method for forming the cell adhesive layer, etc. will be described later in more detail.

(Cell Adhesion Auxiliary Portion)

Hereinafter, the cell adhesion auxiliary portion in the cell culture region in this embodiment is described in detail. The cell adhesion auxiliary portion in this embodiment is not particularly limited insofar as the cell adhesion auxiliary portion is a portion which is formed in a pattern in the cell culture region, inhibits adhesion to cells, and is formed such that, upon adhesion of cells onto the cell adhesion portion, cells on two cell adhesion portions adjacent to the cell adhesion auxiliary portion can be bound to each other on the cell adhesion auxiliary portion.

The cell adhesion auxiliary portion in this embodiment may be, for example, a region where a base material described later is exposed, or may be a region on which a generally used cell adhesion-inhibiting layer etc. inhibiting adhesion to cells is formed. The method for forming the cell adhesion-inhibiting layer includes general printing methods, photolithography method, and patterning methods using the action of a photocatalyst upon irradiation with energy. The patterning methods using the action of a photocatalyst upon irradiation with energy will be described in connection with the cell adhesive layer using a cell adhesion-inhibiting layer containing a cell adhesion-inhibiting material described later, and thus the description thereof is omitted herein.

When the cell adhesive layer is a layer containing the cell adhesive material decomposed or denatured by the action of a photocatalyst upon irradiation with energy as described above, the cell adhesion auxiliary portion may be a region or the like where decomposed or denatured products of the cell adhesive material remain. The method for forming the cell adhesion auxiliary portion in this case will be described in connection with the cell adhesive layer containing the cell adhesive material decomposed by the action of a photocatalyst upon irradiation with energy, and thus its description is omitted herein.

2. Base Material

The following will describe the base material used in this embodiment. The base material used in this embodiment is not particularly limited insofar as it is capable of forming the cell culture region. For example, an inorganic material such as metal, glass and silicon, or an organic material typified by plastic and the like can be used. The flexibility, transparency etc. of the base material are properly selected depending on the type, applications etc. of the cell culture patterning substrate.

In this embodiment, since the region other than the cell culture region on the base material is used as a non-cell culture region on which cells are not cultured, the region preferably inhibits adhesion to cells. For example, a layer or the like inhibiting adhesion to cells may be formed in the non-cell culture region, that is, the region other than the cell culture region.

3. Cell Culture Patterning Substrate

The following will describe the cell culture patterning substrate in this embodiment. The cell culture patterning substrate in this embodiment is not particularly limited insofar as the cell culture region is formed on the above-mentioned base material. If necessary, a member such as a light-shielding portion may be formed thereon.

4. Others

As described above, the cell adhesive layer used in the cell culture region of the cell culture patterning substrate in this embodiment may be: (1) a layer containing a cell adhesive material decomposed or denatured by action of a photocatalyst upon irradiation with energy; or (2) a layer produced by forming a cell adhesion-inhibiting layer containing a cell adhesion-inhibiting material having cell adhesion-inhibiting properties of inhibiting adhesion to cells and also decomposed or denatured by action of a photocatalyst upon irradiation with energy, and then, irradiating it with energy thereby decomposing or denaturing the cell adhesion-inhibiting material.

Hereinafter, each case is described in more detail.

I. Case of (1)

First, the case, wherein the cell adhesive layer contains a cell adhesive material decomposed or denatured by action of a photocatalyst upon irradiation with energy, is described. In respect of the cell adhesive layer containing such a cell adhesive material, there are the following three modes:

In the first mode, the cell adhesive layer is a photocatalyst-containing cell adhesive layer containing a photocatalyst and a cell adhesive material. Upon irradiation of this photocatalyst-containing cell adhesive layer with energy, the cell adhesive material is decomposed or denatured by the action of the photocatalyst contained in the photocatalyst-containing cell adhesive layer itself.

In the second mode, the cell adhesive layer, which contains at least a cell adhesive material, is formed on a photocatalyst treatment layer, which contains at least a photocatalyst. Upon irradiation of the cell adhesive layer with energy, the cell adhesive material in the cell adhesive layer is decomposed and denatured by the action of the photocatalyst contained in the adjacent photocatalyst treatment layer.

In the third mode, the cell adhesive layer, which contains at least a cell adhesive material, is formed on a base material, and upon irradiation with energy, a photocatalyst-containing layer or the like, which contains at least a photocatalyst, is opposed to the cell adhesive layer and then irradiated with energy. Thereby, the cell adhesive material is decomposed or denatured by the action of the photocatalyst in the opposed photocatalyst-containing layer.

Hereinafter, these modes will be described respectively.

(1) First Mode

Now, the case wherein the cell adhesive layer is a photocatalyst-containing cell adhesive layer, which contains a photocatalyst and a cell adhesive material, and upon irradiation of this photocatalyst-containing cell adhesive layer with energy, the cell adhesive material is decomposed or denatured by the action of the photocatalyst contained in the photocatalyst-containing cell adhesive layer itself is described in detail.

According to this mode, since the photocatalyst-containing cell adhesive layer contains a photocatalyst and the above-mentioned cell adhesive material, upon irradiating the photocatalyst-containing cell adhesive layer with energy, the cell adhesive material can be decomposed or denatured by the action of the photocatalyst, and the region irradiated with energy can be made into a cell adhesion auxiliary portion to which cells do not adhere. Since the cell adhesion material remains in the region not irradiated with energy, this region can be made into a cell adhesion portion having excellent cell adhesive properties. Accordingly, the cell adhesion auxiliary portion, which inhibits adhesion to cells, can be easily formed in the cell adhesion portion by irradiation with energy in a pattern, so that a special apparatus or a complicated process is not necessary.

Formation of such a photocatalyst-containing cell adhesive layer can be carried out, for example, by coating a photocatalyst-containing cell adhesive layer forming coating solution, which contains a photocatalyst and a cell adhesive material to be decomposed or denatured by the action of the photocatalyst upon irradiation with energy. The coating of this photocatalyst-containing cell adhesive layer forming coating solution can be carried out by a general coating method, and for example, spin coating, spray coating, dip coating, roll coating, or bead coating can be used.

The thickness of the photocatalyst-containing cell adhesive layer is appropriately selected according to the type of the cell culture patterning substrate and others. Usually, the thickness is about 0.01 μm to 1.0 μm, preferably about 0.1 μm to 0.3 μm.

Hereinafter, the cell adhesive material and the photocatalyst, which are contained in the photocatalyst-containing cell adhesive layer used in this mode, will be described, and the method for forming the cell adhesion auxiliary portion will be described.

a. Cell Adhesive Material

First, a cell adhesive material comprised in the photocatalyst-containing cell adhesive layer of the present mode will be explained. The kind and the like of the cell adhesive material comprised in the photocatalyst-containing cell adhesive layer of the present mode is not particularly limited insofar as it has the cell adhesive properties and can be decomposed or denatured by action of a photocatalyst upon irradiation with energy. Here, “having the cell adhesive properties” means being good in the cell adhesion. For instance, when the cell adhesive properties differ depending on the kind of cells, it means to be good in the adhesion with target cells.

The cell adhesive material used in the present mode has such cell adhesive properties. Those losing the cell adhesive properties or those changed into ones having the cell adhesion-inhibiting properties of inhibiting adhesion to cells, by being decomposed or denatured by the action of the photocatalyst upon irradiation with energy, are used.

As such materials having the cell adhesive properties, there are two kinds, one being materials having the cell adhesive properties owing to physicochemical characteristics and the other being materials having the cell adhesive properties owing to biochemical characteristics.

As physicochemical factors that determine the cell adhesive properties of materials having the cell adhesive properties owing to the physicochemical characteristics, the surface free energy, the electrostatic interaction and the like can be cited. For instance, when the cell adhesive properties is determined by the surface free energy of the material, if the material has the surface free energy in a predetermined range, the adhesive properties between the cells and the material becomes good. If it deviates from the above range the adhesive properties between the cells and material is deteriorated. As such changes of the cell adhesive properties due to the surface free energy, experimental results shown in Data, for instance, CMC Publishing Co., Ltd. “Biomaterial no Saisentan”, Yoshito IKADA (editor), p. 109, lower part are known. As materials having the cell adhesive properties owing to such a factor, for instance, hydrophilic polystyrene, poly(N-isopropyl acrylamide) and the like can be cited. When such a material is used, by the action of the photocatalyst upon irradiation with energy, for instance, a functional group on a surface of the material is substituted, decomposed or the like to cause a change in the surface free energy, resulting in one that does not have the cell adhesive properties or one that has the cell adhesion-inhibiting properties.

When the adhesive properties between cells and a material is determined owing to the electrostatic interaction or the like, for instance, the cell adhesive properties are determined by an amount of positive electric charges and the like that the material has. As materials having the cell adhesive properties owing to such electrostatic interaction, basic polymers such as polylysine; basic compounds such as aminopropyltriethoxysilane, N-(2-aminoethyl)-3-aminopropyltrimethoxysilane; and condensates and the like including these can be cited. When such materials are used, by the action of the photocatalyst upon irradiation with energy, the above-mentioned materials are decomposed or denatured. Thereby, for instance, an amount of positive electric charges present on a surface can be altered, resulting in one that does not have the cell adhesive properties or one that has the cell adhesion-inhibiting properties.

As materials having the cell adhesive properties owing to the biological characteristics, ones that are good in the adhesive properties with particular cells or ones that are good in the adhesive properties with many cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited. When such materials are used, by the action of the photocatalyst upon irradiation with energy, for instance, a structure of the material is partially destroyed, or a principal chain is destroyed or the like, resulting in one that does not have the cell adhesive properties or one that has the cell adhesion-inhibiting properties.

Such a cell adhesive material, though it differs depending on the kind of the materials and the like, is comprised in the photocatalyst-containing cell adhesive layer normally in the range of 0.01% by weight to 95% by weight, and preferably in the range of 1% by weight to 10% by weight. Thereby, a region that contains the cell adhesive material can be made a region good in the cell adhesive properties.

b. Photocatalyst

Next, a photocatalyst comprised in the photocatalyst-containing cell adhesive layer of the present mode will be explained. The photocatalyst used in the present mode is not particularly limited insofar as it can decompose or denature the cell adhesive material described above by the action of the photocatalyst upon irradiation with energy.

Though the action mechanism of a photocatalyst typified by titanium oxide described below is not necessarily clear, it can be considered that a carrier generated by irradiation of light directly reacts with a nearby compound or, owing to an active oxygen species generated under the presence of oxygen, water, a chemical structure of an organic material is caused to be changed. In the present mode, it is considered that this carrier influences the function of the cell adhesive material described above.

As the photocatalyst that can be used in the present mode, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present mode, in particular, titanium dioxide, owing to a large band gap, chemical stability, non-toxicity, and easy availability, can be preferably used. There are two types of titanium dioxide, anatase type and rutile type, and both can be used in the present mode; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

A content of the photocatalyst comprised in the photocatalyst-containing cell adhesive layer of the present mode can be set in the range of 5 to 95% by weight, preferably of 10 to 60% by weight, and more preferably of 20 to 40% by weight. Thereby, a cell adhesive material of the photocatalyst-containing cell adhesive layer in a region where energy is irradiated can be decomposed or denatured.

The photocatalyst used in the present mode, owing to, for instance, high hydrophilicity thereof and the like, is preferably low in the adhesiveness with cells. Thereby, a region where the photocatalyst is exposed, owing to such as the decomposition of a cell adhesive material described above, can be used as a region low in the adhesiveness with the cells.

c. Others

In this mode, not only the cell adhesive material and the photocatalyst but also a binder etc. for improving strength, resistance etc. may be contained as necessity in the photocatalyst-containing cell adhesive layer. In the present mode, particularly as the binder, a material that, at least after the energy irradiation, has the cell adhesion inhibiting properties of inhibiting adhesion to cells is preferably used. This is because the adhesion between cells and the cell adhesion auxiliary portion, which is a region irradiated with energy, can thereby be reduced. As such a material, one that has the cell adhesion inhibiting properties prior to the energy irradiation or one that obtains the cell adhesion inhibiting properties by the action of the photocatalyst upon irradiation with energy may be used.

In the present mode, a material that becomes to have the cell adhesion inhibiting properties, particularly by the action of the photocatalyst upon irradiation with energy, is preferably used as a binder. Thereby, in a region prior to the energy irradiation, the adhesiveness between the cell adhesive material and cells is not inhibited, and only a region where energy is irradiated can be lowered in the adhesiveness with the cells.

As materials that can be used as such a binder, for instance, ones in which a main skeleton has such a high bond energy that cannot be decomposed by the photo-excitation of the photocatalyst and an organic substituent can be decomposed by an action of the photocatalyst are preferably used. For instance, (1) organopolysiloxane that exhibits large strength by hydrolyzing or polycondensating chloro- or alkoxysilane or the like owing to a sol-gel reaction and the like, and (2) organopolysiloxane and the like in which reactive silicones excellent in the water repellency or oil repellency are crosslinked can be cited.

In the case of the (1), it is preferable to be organopolysiloxanes that are hydrolysis condensates or cohydrolysis condensates of at least one kind of silicon compounds expressed by a general formula:
YₙSiX₍₄₋ₙ₎
(Here, Y denotes an alkyl group, fluoroalkyl group, vinyl group, amino group, phenyl group, epoxy group or organic group containing the above, and X denotes an alkoxyl group, acetyl group or halogen. “n” is an integer of 0 to 3.). The number of carbons of the group expressed with Y is preferably in the range of 1 to 20, and the alkoxy group shown with X is preferably a methoxy group, ethoxy group, propoxy group or butoxy group.

As the reactive silicone according to the (2), compounds having a skeleton expressed by a general formula below can be cited. 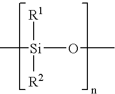
In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R² is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material such as mentioned above is used, by the action of the photocatalyst upon irradiation with energy, a surface of an energy-irradiated region can be made high in the hydrophilicity. Thereby, the adhesion with cells is inhibited, and the region where energy is irradiated can be made into a region on which the cells do not adhere.

In the case of using the above-mentioned material as the cell adhesion-inhibiting material, the contact angle thereof with water is preferably in the range of 15° to 120°, more preferably in the range of 20° to 100° before the material is irradiated with energy. According to this, the cell adhesive properties can be rendered good.

In the case of irradiating this cell adhesion-inhibiting material with energy, it is preferred that the contact angle thereof with water becomes 10° or less. This range makes it possible to render the material having a high hydrophilicity and low cell adhesive properties.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

Together with the organopolysiloxanes, a stable organo silicium compound that does not cause a crosslinking reaction, such as dimethylpolysiloxanes, may be blended with a binder.

In the present mode, a decomposition substance or the like that causes such as a change in the wettability of a region where energy is irradiated, thereby lowers the adhesiveness with cells or that aides such a change may be contained.

As such decomposition substances, for instance, surfactants or the like that are decomposed and the like, by the action of the photocatalyst upon irradiation with energy, to be hydrophilic and the like to result in lowering the adhesiveness with cells can be cited. Specifically, nonionic surfactants: hydrocarbon based such as respective series of NIKKOL BL, BC, BO, and BB manufactured by Nikko Chemicals Co., Ltd.; and silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNE DS-401 and 402 manufactured by DAIKIN INDUSTRIES, Ltd., and Fluorad FC-170 and 176 manufactured by 3M can be cited. Cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxyresin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present mode, such a binder can be preferably comprised in the photocatalyst-containing cell adhesive layer, in the range of 5% by weight to 95% by weight, more preferably 40% by weight to 90% by weight, and particularly preferably 60% by weight to 80% by weight.

In this mode, a light-shielding portion may be formed if necessary on the cell culture region of the base material. This is because when the whole surface of the photocatalyst-containing cell adhesive layer on the base material is irradiated with energy from the base material side, the photocatalyst on the region provided with the light-shielding portion is not excited, so the cell adhesive material contained in the region of the cell adhesive layer other than the region provided with the light-shielding portion can be decomposed or denatured.

The light-shielding portion is not particularly limited insofar as it can shield energy that is irradiated on forming the cell adhesion auxiliary portion. For instance, the light-shielding portion may be formed by forming a metal thin film made of chromium or the like into a thickness of about 1000 to 2000 Å by a sputtering method, a vacuum deposition method or the like, and then, patterning the thin film. As the patterning method, an ordinary patterning method such as the sputtering can be used.

A method may be one by which a layer that contains light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds, and furthermore a photosensitive resin and an O/W emulsion type resin composition such as emulsified reactive silicone can be used. A thickness of such the resinous light-shielding portion can be set in the range of 0.5 to 10 μm. As a method for patterning such the resinous light-shielding portion, methods such as a photolithography method and a printing method that are generally used can be used.

d. Method for Forming Cell Adhesion Auxiliary Portion

Now, the method for forming the cell adhesion auxiliary portion in this mode is described in detail. In this mode, as shown in FIG. 6 for example, a photocatalyst-containing cell adhesive layer 7 containing the cell adhesive material and the photocatalyst is irradiated, using a photomask 5 or the like, with energy 6 in a pattern to form a cell adhesion auxiliary portion (FIG. 6A), whereby the cell adhesive material can be decomposed or denatured to form a cell adhesion auxiliary portion 4, which inhibits adhesion to cells, in the cell adhesive layer 7 (FIG. 6B). In this case, the cell adhesion auxiliary portion contains the photocatalyst and decomposed or denatured products of the cell adhesive material.

The energy irradiation (exposure) mentioned in this mode is a concept that includes all energy ray irradiation that can decompose or denature the cell adhesive material by the action of the photocatalyst upon irradiation with energy, and is not limited to light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which pattern irradiation is carried out via a photomask by using the above-mentioned light source, a method of carrying out drawing irradiation in a pattern by using laser such as excimer, YAG and the like can be applied. Furthermore, as mentioned above, when the base material has the light-shielding portion in a pattern same as that of the cell adhesion portion, energy can be irradiated over the entire surface from the base material side. In this case, there are advantages in that there are no needs of the photomask and the like and a process of positional alignment and the like.

An amount of irradiation of energy at the energy irradiation is an amount of irradiation necessary for decomposing or denaturing the cell adhesive material by the action of the photocatalyst.

At this time, by irradiating a layer containing the photocatalyst, with energy, while heating, the sensitivity can be raised; accordingly, it is preferable in that the cell adhesive material can be efficiently decomposed or denatured. Specifically, it is preferable to heat in the range of 30° C. to 80° C.

The energy irradiation that is carried out via a photomask in this mode, when the above-mentioned base material is transparent, may be carried out from either direction of the base material side or a photocatalyst-containing cell adhesive layer side. On the other hand, when the base material is opaque, it is necessary to irradiate energy from a photocatalyst-containing cell adhesive layer side.

(2) Second Mode

Now, the case, wherein the cell adhesive layer containing at least a cell adhesive material is formed on a photocatalyst treatment layer containing at least a photocatalyst, and upon irradiation of the cell adhesive layer with energy, the cell adhesive material in the cell adhesive layer is decomposed or denatured by the action of the photocatalyst in the adjacent photocatalyst treatment layer, is described in detail.

In this mode, since the cell adhesive layer is formed on the photocatalyst treatment layer, by irradiating with energy in a pattern of the cell adhesion auxiliary portion to be formed, the cell adhesive material in the cell adhesive layer can be decomposed or denatured by the action of the photocatalyst in the adjacent photocatalyst treatment layer so as to lower the cell adhesive properties of that region. Thereby, the region can be used as the cell adhesion auxiliary portion. In this case, when the cell adhesive material is decomposed by the action of the photocatalyst upon irradiation with energy, the cell adhesion auxiliary portion contains a small amount of the cell adhesive material or decomposed products of the cell adhesive material. Otherwise, the cell adhesive layer is completely decomposed and removed to expose the photocatalyst treatment layer. When the cell adhesive material is denatured by the action of the photocatalyst upon irradiation with energy, its denatured products are contained in the cell adhesion auxiliary portion.

Hereinafter, the cell adhesive layer and the photocatalyst treatment layer used in this mode are described in detail. The method for forming the cell adhesion auxiliary portion in this mode is the same as in the first mode described above, and thus its description is omitted herein.

a. Cell Adhesive Layer

First, the cell adhesive layer used in this mode is described. The cell adhesive layer used in this mode is a layer having at least a cell adhesive material having adhesion to cells, and generally a layer used as a layer having cell adhesive properties can be used.

As the specific cell adhesive material, the same cell adhesive material used in the photocatalyst-containing cell adhesive layer described in the first mode can be used. Thus, its detailed description is omitted. Preferably, the cell adhesive layer in this mode also contains the material having cell adhesion-inhibiting properties described in the photocatalyst-containing cell adhesive layer in the first mode. The cell adhesive properties of the cell adhesion auxiliary portion, which is the energy-irradiated region, can thereby be decreased.

Formation of the cell adhesive layer can be carried out by coating a cell adhesive layer forming coating solution containing the cell adhesive material by a general coating method. Since it can be carried out by the same method for forming the photocatalyst-containing cell adhesive layer in the first mode, its description is omitted.

The thickness of the cell adhesive layer is suitably selected depending on the type and the like of the cell culture patterning substrate. Usually, the thickness may be about 0.001 μm to 1.0 μm, preferably about 0.005 μm to 0.3 μm.

b. Photocatalyst Treatment Layer

Now, the photocatalyst treatment layer used in this mode is described. The photocatalyst treatment layer used in this mode is not particularly limited insofar as it is a layer containing at least a photocatalyst. The photocatalyst treatment layer may be a layer consisting of a photocatalyst only or may be a layer containing other component such as a binder.

The photocatalyst used in this mode can be the same as in the photocatalyst-containing cell adhesive layer in the first mode. The titanium oxide is also particularly preferably used in this mode.

The photocatalyst treatment layer consisting of a photocatalyst only is advantageous in costs because the efficiency of decomposing or denaturing the cell adhesive material in the cell adhesive layer is improved to reduce the treatment time. On the other hand, use of the photocatalyst treatment layer comprising a photocatalyst and a binder is advantageous in that the photocatalyst treatment layer can be easily formed.

An example of the method for forming the photocatalyst treatment layer made only of a photocatalyst may be a vacuum film-forming method such as sputtering, CVD or vacuum vapor deposition. The formation of the photocatalyst treatment layer by the vacuum film-forming method makes it possible to render the layer a homogeneous photocatalyst treatment layer made only of a photocatalyst. Thereby, the cell adhesive material can be decomposed or denatured homogeneously. At the same time, since the layer is made only of a photocatalyst, the cell adhesive material can be decomposed or denatured more effectively, as compared with the case of using a binder.

Another example of the method for forming the photocatalyst treatment layer made only of a photocatalyst, is the following method: for example, in the case that the photocatalyst is titanium dioxide, amorphous titania is formed on the base material, and then, calcinating so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is calcinated at 400° C. to 500° C. so as to be denatured to anatase type titania, and calcinated at 600° C. to 700° C. so as to be denatured to rutile type titania.

In the case of using a binder, the binder preferably having a high bonding energy, wherein its main skeleton is not decomposed by photoexcitation of the photocatalyst. Examples of such a binder include the organopolysiloxanes described in the above-mentioned item “Cell Adhesive Layer”.

In the case of using such an organopolysiloxane as the binder, the photocatalyst treatment layer can be formed by dispersing a photocatalyst, the organopolysiloxane as the binder, and optional additives if needed into a solvent to prepare a coating solution, and coating this coating solution onto the base material. The used solvent is preferably an alcoholic based organic solvent such as ethanol or isopropanol. The coating can be performed by a known coating method such as spin coating, spray coating, dip coating, roll coating, or bead coating. When the coating solution contains an ultraviolet curable component as the binder, the photocatalyst treatment layer can be formed by curing the coating solution through the irradiation of ultraviolet rays.

As the binder, an amorphous silica precursor can be used. This amorphous silica precursor is preferably a silicon compound represented by the general formula SiX₄, wherein X are a halogen, a methoxy group, an ethoxy group, an acetyl group or the like; a silanol which is a hydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst treatment layer can be formed by dispersing the amorphous silica precursor and particles of a photocatalyst homogeneously into a non-aqueous solvent, hydrolyzing with water content in the air to form a silanol onto a transparent base material, and then subjecting to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content of the photocatalyst in the photocatalyst treatment layer can be set in the range of 5 to 60% by weight, preferably in the range of 20 to 40% by weight. The thickness of the photocatalyst treatment layer is preferably in the range of 0.05 to 10 μm.

Besides the above-mentioned photocatalyst and binder, the surfactant and so on used in the above-mentioned cell adhesive layer can be incorporated into the photocatalyst treatment layer.

In the present mode, it is preferred that the surface of the photocatalyst treatment layer is low in cell adhesive properties by having, for example, hydrophilicity for the following reason: this makes it possible that when the cell adhesive layer is decomposed and the like to make the photocatalyst treatment layer exposed, the exposed region is rendered a region low in cell adhesive properties.

In the present mode, one or more light-shielding portions may be formed on the photocatalyst treatment layer, as described above. According to this, when the entire surface of the cell adhesive layer is irradiated with energy, photocatalyst in the regions on which the light-shielding portions are formed are not excited, so that the cell adhesive material, contained in regions of the cell adhesive layer other than the regions thereof on which the light-shielding portions are formed, can be decomposed or denatured. This case has an advantage that the direction in which the energy is irradiated is not particularly limited since the photocatalyst in the regions where the light-shielding portions are formed is not excited.

The light-shielding portion used can be the same as described in the first mode, and thus its detailed description is omitted.

(3) Third Mode

Now, the case, wherein the cell adhesive layer containing at least a cell adhesive material is formed on a base material, and upon irradiation with energy, a photocatalyst-containing layer or the like containing at least a photocatalyst is opposed to the cell adhesive layer, and then, irradiated with energy thereby decomposing or denaturing the cell adhesive material by the action of the photocatalyst in the opposite photocatalyst-containing layer, is described.

In this mode, the cell adhesive layer and the photocatalyst-containing layer are arranged to be opposite to each other and irradiated with energy in a pattern of the cell adhesion auxiliary portion to be formed, whereby the cell adhesive material in the cell adhesive layer can be decomposed or denatured by the action of the photocatalyst in the photocatalyst-containing layer to form a cell adhesion auxiliary portion.

Hereinafter, the photocatalyst-containing layer side substrate used in this mode, and the method for forming the cell adhesion auxiliary portion by using the photocatalyst-containing layer side substrate, are described. The cell adhesive layer used in this mode is the same as the cell adhesive layer used in the second mode described above, and thus its description is omitted.

a. Photocatalyst-Containing Layer Side Substrate

First, the photocatalyst-containing layer side substrate, comprising a photocatalyst-containing layer containing a photocatalyst, used in this mode is described. The photocatalyst-containing layer side substrate used in this mode is usually a substrate comprising a photocatalyst-containing layer containing a photocatalyst. Usually, it comprises a base body and a photocatalyst-containing layer formed on the base body. This photocatalyst-containing layer side substrate may have, for example, photocatalyst-containing layer side light-shielding portions formed in a pattern, a primer layer, or the like. The following will describe each of the constituents of the photocatalyst-containing layer side substrate used in this mode.

(i) Photocatalyst-Containing Layer

First, the photocatalyst-containing layer used in the photocatalyst-containing layer side substrate is described. The photocatalyst-containing layer used in this mode is not particularly limited insofar as the layer is constituted such that the photocatalyst in the photocatalyst-containing layer can cause the decomposition or denaturation of the cell adhesive material in the adjacent cell adhesive layer. The photocatalyst-containing layer may be composed of a photocatalyst and a binder, or may be made only of a photocatalyst. The property of the surface thereof may be lyophilic or repellent to liquid.

The photocatalyst-containing layer used in this mode may be formed on the whole surface of a base body, or as shown in FIG. 7 for example, a photocatalyst-containing layer 12 may be formed in a pattern on a base body 11.

By forming the photocatalyst-containing layer in a pattern as described above, patterning irradiation via a photomask or the like is not necessary at the time of irradiating energy to form a cell adhesion auxiliary portion. Therefore, by irradiating the whole surface, the cell adhesion auxiliary portion, wherein the cell adhesive material contained in the cell adhesive layer is decomposed or denatured, can be formed.

The method for patterning the photocatalyst-containing layer is not particularly limited. For example, a method such as a photolithography may be used.

Only on the area of the cell adhesive layer which actually faces the photocatalyst-containing layer, the cell adhesive material is decomposed or denatured. Therefore, the direction in which energy is irradiated may be any direction if the energy is irradiated onto the area where the photocatalyst-containing layer and the cell adhesive layer facing to each other. Further, there is an advantage that the irradiated energy is not particularly limited to energy composed of parallel constituents, such as parallel light.

The photocatalyst-containing layer used in this mode can be the same as the photocatalyst treatment layer described above in the second mode, and thus its detailed description is omitted.

(ii) Base Body

The following will describe the base body used in the photocatalyst-containing layer side substrate. Usually, the photocatalyst-containing layer side substrate has at least a base body and a photocatalyst-containing layer formed on the base body. In this case, the material which constitutes the base body to be used is appropriately selected depending on the direction of energy irradiation which will be detailed later, necessity of the resulting pattern-forming body to be transparency, or other factors.

The base body used in this mode may be a member having flexibility, such as a resin film, or may be a member having no flexibility, such as a glass substrate. This is appropriately selected depending on the method for the energy irradiation.

An anchor layer may be formed on the base body in order to improve the adhesion between the surface of the base body and the photocatalyst-containing layer. The anchor layer may be made of, for example, a silane based or titanium based coupling agent.

(iii) Photocatalyst-Containing Layer Side Light-Shielding Portion

The photocatalyst-containing layer side substrate used in this mode may be a photocatalyst-containing layer side substrate on which photocatalyst-containing layer side light-shielding portions are formed in a pattern. When the photocatalyst-containing layer side substrate having photocatalyst-containing layer side light-shielding portions is used in this way, at the time of irradiating energy, it is not necessary to use any photomask or to carry out drawing irradiation with a laser light. Since alignment of the photomask and the photocatalyst-containing layer side substrate is not necessary, process can be made simple. Further, since expensive device for drawing irradiation is also not necessary, it is advantageous in costs.

Such a photocatalyst-containing layer side substrate having photocatalyst-containing layer side light-shielding portions can be classified into the following two embodiments, depending on the position where the photocatalyst-containing layer side light-shielding portions are formed.

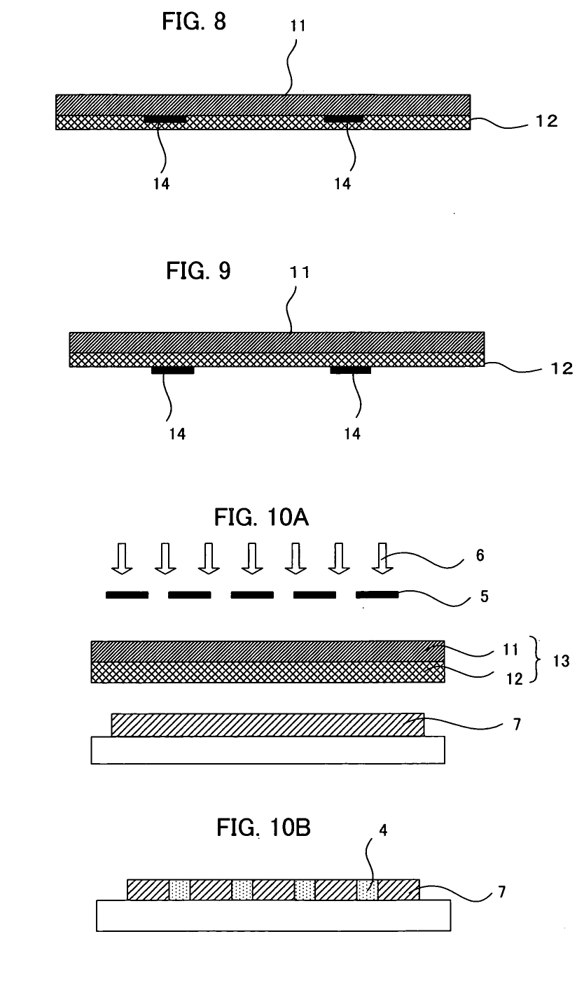

One of them is an embodiment, as shown in FIG. 8 for example, wherein photocatalyst-containing layer side light-shielding portions 14 are formed on a base body 11, and a photocatalyst-containing layer 12 is formed on the photocatalyst-containing layer side light-shielding portions 14 to obtain the photocatalyst-containing layer side substrate. The other example is an embodiment, as shown in FIG. 9 for example, wherein a photocatalyst-containing layer 12 is formed on a base body 11, and photocatalyst-containing layer side light-shielding portions 14 are formed thereon to obtain the photocatalyst-containing layer side substrate.

In any one of the embodiments, since the photocatalyst-containing layer side light-shielding portions are arranged near the region where the photocatalyst-containing layer and the cell adhesive layer are arranged, the effect of energy-scattering in the base body or the like can be made smaller than in the case of using a photomask. Accordingly, irradiation of energy in a pattern can be more precisely attained.

In this mode, in the case of the embodiment, wherein photocatalyst-containing layer side light-shielding portions 14 are formed on a photocatalyst-containing layer 12 as shown in FIG. 9, there is an advantage that at the time of arranging the photocatalyst-containing layer and the cell adhesive layer in a predetermined position, the photocatalyst-containing layer side light-shielding portions can be used as a spacer for making the interval constant, by making the film thickness of the photocatalyst-containing layer side light-shielding portions consistent with the width of the interval between the two layers.

In other words, when the photocatalyst-containing layer and the cell adhesive layer are arranged so as to be facing each other at a predetermined interval, by arranging the photocatalyst-containing layer side light-shielding portions and the cell adhesive layer in close contact to each other, the dimension of the predetermined interval can be made precise. When energy is irradiated in this state, cell adhesion auxiliary portions can be formed with a good precision since cell adhesive material is not decomposed or denatured in the cell adhesive layer inside the region where the cell adhesive layer and the light-shielding portions are in contact.

The method for forming such photocatalyst-containing layer side light-shielding portions is not particularly limited, and may be appropriately selected in accordance with the property of the surface on which the photocatalyst-containing layer side light-shielding portions are to be formed, shielding ability against the required energy, and others. The light-shielding portions may be the same light-shielding portions as described in the first mode which are formed on a base material. Thus, the detailed description thereof is omitted herein.

The above has described two cases, wherein the photocatalyst-containing layer side light-shielding portions are formed between the base body and the photocatalyst-containing layer and are formed on the surface of the photocatalyst-containing layer. Besides, the photocatalyst-containing layer side light-shielding portions may be formed on the base body surface of the side on which the photocatalyst-containing layer is not formed. In this embodiment, for example, a photomask can be made in close contact to this surface to such a degree that the photomask in removable. Thus, this embodiment can be preferably used for the case that the pattern of the cell adhesion auxiliary portions is changed for every small lot.

(iv) Primer Layer

The following will describe a primer layer used in the photocatalyst-containing layer side substrate of this mode. In this mode, when photocatalyst-containing layer side light-shielding portions are formed into a pattern on a base body and a photocatalyst-containing layer is formed thereon so as to prepare a photocatalyst-containing layer side substrate described above, a primer layer may be formed between the photocatalyst-containing layer side light-shielding portions and the photocatalyst-containing layer.

The effect and function of this primer layer are not necessarily clear, but would be as follows: by forming the primer layer between the photocatalyst-containing layer side light-shielding portions and the photocatalyst-containing layer, the primer layer is assumed to exhibit a function of preventing the diffusion of impurities from the photocatalyst-containing layer side light-shielding portion and openings present between the photocatalyst-containing layer side light-shielding portions, in particular, residues generated when the photocatalyst-containing layer side light-shielding portions are patterned, or metal or metal ion impurities; the impurities being factors for blocking the decomposition or denaturation of the cell adhesive material by action of the photocatalyst. Accordingly, by forming the primer layer, it is possible to process the decomposition or denaturation of the cell adhesive material with high sensitivity, so as to yield cell adhesion auxiliary portions which are highly precisely formed.

The primer layer in this mode is a layer for preventing the effect of the photocatalyst from being affected by the impurities present in not only the photocatalyst-containing layer side light-shielding portions but also in the openings formed between the photocatalyst-containing layer side light-shielding portions. It is therefore preferred to form the primer layer over the entire surface of the photocatalyst-containing layer side light-shielding portions including the openings.

The primer layer in this mode is not particularly limited insofar as the primer layer is formed not to bring the photocatalyst-containing layer side light-shielding portions and the photocatalyst-containing layer of the photocatalyst-containing layer side substrate into contact with each other.

A material that forms the primer layer, though not particularly limited, is preferably an inorganic material that is not likely to be decomposed by the action of the photocatalyst. Specifically, amorphous silica can be cited. When such amorphous silica is used, a precursor of the amorphous silica is preferably a silicon compound that is represented by a general formula, SiX₄, wherein X being halogen, methoxy group, ethoxy group, acetyl group or the like; silanol that is a hydrolysate thereof, or polysiloxane having an average molecular weight of 3000 or less.

A film thickness of the primer layer is preferably in the range of 0.001 μm to 1 μm and particularly preferably in the range of 0.001 μm to 0.1 μm.

b. Method for Forming Cell Adhesion Auxiliary Portion

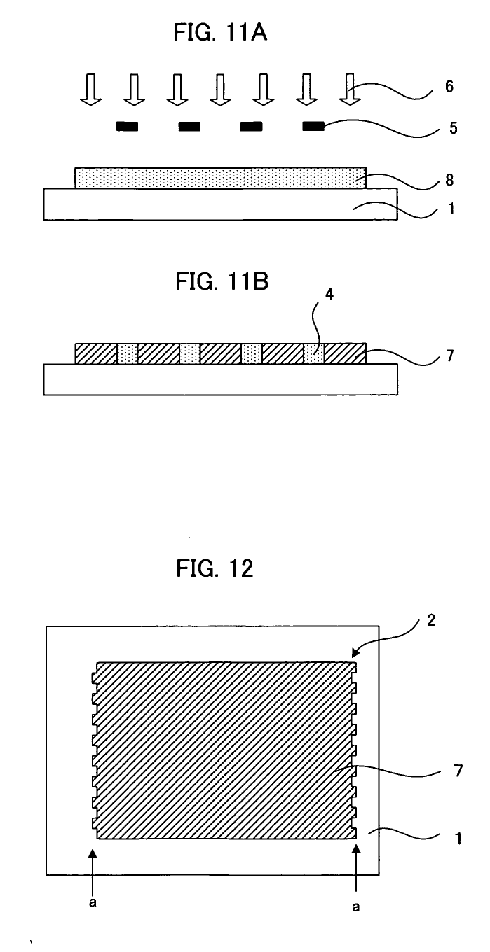

Hereinafter, the method for forming the cell adhesion auxiliary portion in this mode is described. In this mode, for example as shown in FIG. 10, a cell adhesive layer 7 and a photocatalyst-containing layer 12 of a photocatalyst-containing layer side substrate 13 are arranged with a predetermined space, and irradiated with energy 6 from a predetermined direction for example via photomask 5 or the like. The cell adhesive material in the region irradiated with energy is thereby decomposed or denatured, whereby the cell adhesion auxiliary portion 4 inhibiting adhesion to cells is formed in the cell adhesive layer 7. In this case, when the cell adhesive material is decomposed for example by the action of a photocatalyst upon irradiation with energy, the cell adhesion auxiliary portion contains a small amount of the cell adhesive material or decomposed products of the cell adhesive material. Otherwise, the cell adhesive layer is completely decomposed and removed to expose the base material. When the cell adhesive material is denatured by the action of a photocatalyst upon irradiation with energy, its denatured products are contained in the cell adhesion auxiliary portion.

The above-mentioned wording “arranging” means that the above-mentioned two layers; the photocatalyst-containing layer and the cell adhesive layer are arranged in the state that the action of the photocatalyst can substantially work to the surface of the cell adhesive layer, and include not only the state that the two layers actually contact each other, but also the state that the two layers are arranged at a predetermined interval. The dimension of the interval is preferably 200 μm or less.

In this mode, the dimension of the above-mentioned interval is more preferably in the range of 0.2 μm to 10 μm, even more preferably in the range of 1 μm to 5 μm, since the precision of the pattern to be obtained becomes very good and further the sensitivity of the photocatalyst becomes high so as to make good efficiency of the decomposition or denaturation of the cell adhesive material in the cell adhesive layer. This range of the interval dimension is particularly effective for the cell adhesive layer which is small in area, wherein the interval dimension can be controlled with a high precision.

Meanwhile, in the case of treating the cell adhesive layer large having area, for example, 300 mm×300 mm or more in size, it is very difficult to make a fine interval as described above between the photocatalyst-containing layer side substrate and the cell adhesive layer without contacting each other. Accordingly, when the cell adhesive layer has a relatively large area, the interval dimension is preferably in the range of 10 to 100 μm, more preferably in the range of 50 to 75 μm. By setting the interval dimension in the above range, problems will not occur that: deterioration of patterning precision, such as blurring of the pattern; or the sensitivity of the photocatalyst deteriorates so that the efficiency of decomposing or denaturing the cell adhesive material is also deteriorated. Further, there is an advantageous effect that the cell adhesive material is not unevenly decomposed or denatured.

When energy is irradiated onto the cell adhesive material having a relatively large area as described above, the dimension of the interval, in a unit for positioning the photocatalyst-containing layer side substrate and the cell adhesive layer inside the energy irradiating device, is preferably set in the range of 10 μm to 200 μm, more preferably in the range of 25 μm to 75 μm. The setting of the interval dimension value into this range makes it possible to arrange the photocatalyst-containing layer side substrate and the cell adhesive layer without causing a large deterioration of patterning precision or of sensitivity of the photocatalyst, or bringing the substrate and the layer into contact with each other.

When the photocatalyst-containing layer and the surface of the cell adhesive layer are arranged at a predetermined interval as described above, active oxygen species generated from oxygen and water by action of the photocatalyst can easily be released. In other words, if the interval between the photocatalyst-containing layer and the cell adhesive layer is made narrower than the above-mentioned range, the active oxygen species are not easily released, so as to make the rate for decomposing or denaturing the cell adhesive material unfavorably small. If the two layers are arranged at an interval larger than the above-mentioned range, the generated active oxygen species do not reach the cell adhesive layer easily. In this case also, the rate for decomposing or denaturing the cell adhesive material unfavorably becomes unfavorably small.

The method for arranging the photocatalyst-containing layer and the cell adhesive layer to make such a very small interval evenly therebetween is, for example, a method of using spacers. The use of the spacers in this way makes it possible to make an even interval. At the same time, the action of the photocatalyst does not work onto the surface of the cell adhesive layer in the regions which the spacers contact. Therefore, when the spacers are rendered to have a pattern similar to that of the cell adhesion portions, the cell adhesive material only inside regions where no spacers are formed can be decomposed or denatured so that highly precise cell adhesion-inhibiting portions can be formed. The use of the spacers also makes it possible that the active oxygen species generated by action of the photocatalyst reach the surface of the cell adhesive layer, without diffusing, at a high concentration. Accordingly, highly precise cell adhesion auxiliary portions can be effectively formed.

In this mode, it is sufficient that such an arrangement state of the photocatalyst-containing layer side substrate is maintained only during the irradiation of energy.

The energy irradiation (exposure) mentioned in this mode is a concept that includes all energy ray irradiation that can decompose or denature the cell adhesive material by the action of the photocatalyst upon irradiation with energy, and is not limited to light irradiation.

The type etc. of irradiated energy in this mode is the same as in the first mode described above, and thus their detailed description is omitted herein.

The energy irradiation direction that is carried out via a photomask in this mode, when the above-mentioned base material is transparent, may be carried out from either direction of the side of the base material or the side of the photocatalyst-containing layer side substrate. On the other hand, when the base material is opaque, it is necessary to irradiate energy from the side of the photocatalyst-containing layer side substrate.

II. Case of (2)

Now, the case, where the cell adhesive layer is a layer obtained by forming a cell adhesion-inhibiting layer containing a cell adhesion-inhibiting material having cell adhesion-inhibiting properties of inhibiting adhesion to cells and decomposed or denatured by the action of a photocatalyst upon irradiation with energy, and then, irradiating it with energy to decompose or denature the cell adhesion-inhibiting material, will be described in detail. In this case, the following three modes are mentioned.

In the first mode, the cell adhesion-inhibiting layer is a photocatalyst-containing cell adhesion-inhibiting layer containing a photocatalyst and a cell adhesion-inhibiting material inhibiting adhesion to cells. This photocatalyst-containing cell adhesion-inhibiting layer is irradiated with energy in a pattern of a cell adhesive layer to be formed, thereby obtaining a cell adhesion layer wherein the cell adhesion-inhibiting material is decomposed or denatured by the action of the photocatalyst contained in the photocatalyst-containing cell adhesion-inhibiting layer itself.

In the second mode, the cell adhesion-inhibiting layer containing at least a cell adhesion-inhibiting material is formed on a photocatalyst treatment layer containing at least a photocatalyst, and this cell adhesion-inhibiting layer is irradiated with energy in a pattern of a cell adhesive layer to be formed, thereby obtaining a cell adhesion layer wherein the cell adhesion-inhibiting material is decomposed and denatured by the action of the photocatalyst contained in the photocatalyst treatment layer.

In the third mode, the cell adhesion-inhibiting layer containing at least a cell adhesion-inhibiting material is formed on a base material, and the photocatalyst-containing layer, etc. containing at least a photocatalyst are opposed to the cell adhesion-inhibiting layer and then irradiated with energy in a pattern of a cell adhesive layer to be formed, thereby obtaining a cell adhesive layer wherein the cell adhesion-inhibiting material is decomposed or denatured.

Hereinafter, these modes are described respectively.

(1) First Mode

Now, the mode, wherein the cell adhesion-inhibiting layer is a photocatalyst-containing cell adhesion-inhibiting layer containing a photocatalyst and a cell adhesion-inhibiting material inhibiting adhesion to cells, and this photocatalyst-containing cell adhesion-inhibiting layer is irradiated with energy in a pattern of a cell adhesive layer to be formed, thereby obtaining a cell adhesion layer in which the cell adhesion-inhibiting material is decomposed or denatured by the action of the photocatalyst contained in the photocatalyst treatment layer, is described.

According to this mode, since the photocatalyst-containing cell adhesion-inhibiting layer contains a photocatalyst and the above-mentioned cell adhesion-inhibiting material, by irradiating the photocatalyst-containing cell adhesion-inhibiting layer with energy, the cell adhesion-inhibiting material can be decomposed or denatured by the action of the photocatalyst. Thus, the region irradiated with energy can serve as a cell adhesion portion having cell adhesive properties, that is, a cell adhesive layer. The region not irradiated with energy can serve as a cell adhesion auxiliary portion.

Formation of such a photocatalyst-containing cell adhesion-inhibiting layer can be conducted for example by coating a photocatalyst-containing cell adhesion-inhibiting layer-forming coating solution, containing a photocatalyst and a cell adhesive material to be decomposed or denatured by the action of the photocatalyst upon irradiation with energy, onto the cell culture region. Coating of the photocatalyst-containing cell adhesion-inhibiting layer-forming coating solution can be carried out by a general coating method. For example, spin coating, spray coating, dip coating, roll coating, or bead coating can be used.

The thickness of the photocatalyst-containing cell adhesion-inhibiting layer is appropriately selected according to the type of the cell culture patterning substrate and others, and is usually about 0.01 μm to 1.0 μm, preferably about 0.1 μm to 0.3 μm.

Hereinafter, the cell adhesion-inhibiting material is described, and further, the method for forming the cell adhesive layer is described. The photocatalyst used in this mode can be the same as the photocatalyst used in the first mode in “I. Case of (1)” described above, and thus its detailed description is omitted herein.

a. Cell Adhesion-Inhibiting Material

First, the cell adhesion-inhibiting material contained in the photocatalyst-containing cell adhesion-inhibiting layer used in this mode is described.

The type etc. of the cell adhesion-inhibiting material used in this mode are not particularly limited insofar as the cell adhesion-inhibiting material has cell adhesion-inhibiting properties of inhibiting adhesion to cells and is decomposed or denatured by the action of a photocatalyst upon irradiation with energy.

The phrase “to have cell adhesion-inhibiting properties” means to have a property of preventing cells from being adhered to the cell adhesion-inhibiting material, and when the cell adhesive properties varies depending on the type of the cell, the phrase means to have a property of inhibiting adhesion with the objective cells.

The cell adhesion-inhibiting material used in this mode is a material having such cell adhesion-inhibiting properties. A material which loses the cell adhesion-inhibiting properties or which obtains good cell adhesive properties, by being decomposed or denaturated by the action of a photocatalyst upon irradiation with energy, is used.

As the cell adhesion-inhibiting material, a material having high hydration ability can be used. The material having high hydration ability forms a hydration layer wherein water molecules gather around thereof. Usually, since such a material having high hydration ability has higher adhesion to water molecules than adhesion to cells, the cells can not be adhered to the material having high hydration ability. Thus, the layer will have poor cell adhesive properties. The hydration ability is referred to as a property of hydrating with water molecules, and high hydration ability is intended to mean that the material is easily hydrated with water molecules.

As the material having high hydration ability which is used as a cell adhesion-inhibiting material, for example, polyethylene glycol, amphoteric ionic materials having a betaine structure, phospholipid-containing materials, etc can be listed. When such materials are used as the cell adhesion-inhibiting material, upon irradiated with energy in the below-described energy irradiating process, the cell adhesion-inhibiting material is decomposed or denatured by the action of a photocatalyst so as to remove the hydration layer on the surface, thereby obtaining the material not having the cell adhesion-inhibiting properties.

In this mode, a surfactant, which is decomposed by the action of a photocatalyst and has water repellent or oil repellent organic substituent, can also be used as the cell adhesion-inhibiting material. As such surfactant for example, nonionic surfactants such as: hydrocarbon based such as the respective series of NIKKOL BL, BC, BO, and BB manufactured by Nikko Chemicals Co., Ltd.; and fluorine based or silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F251 manufactured by NEOS, UNIDYNE DS-401 and 402 manufactured by DAIKIN INDUSTRIES, Ltd., and Fluorad FC-170 and 176 manufactured by 3M can be cited. Also, cationic surfactants, anionic surfactants and amphoteric surfactants also can be used.

When the photocatalyst-containing cell adhesion-inhibiting layer is formed by using the above material as the cell adhesion-inhibiting material, the cell adhesion-inhibiting material is unevenly distributed on the surface. The water repellency or oil repellency can thereby be increased, and the interaction with cells can be decreased to reduce cell adhesive properties. Upon irradiation of this layer with energy in the energy irradiating process, the material is easily decomposed by the action of the photocatalyst to expose the photocatalyst. Thus, one not having the cell adhesion-inhibiting properties can be obtained.

In this mode, a material, which obtains good cell adhesive properties by the action of the photocatalyst upon irradiation with energy, is particularly preferably used as the cell adhesion-inhibiting material. As such cell adhesion-inhibiting material, for example, materials having oil repellency or water repellency can be listed.

When the material having oil repellency or water repellency is used as the cell adhesion-inhibiting material, the interaction such as hydrophobic interaction between the cells and the cell adhesion-inhibiting material is made low by the water repellency or oil repellency of the cell adhesion-inhibiting material, thereby decreasing cell adhesive properties.

As the material having water repellency or oil repellency is, a material, for example, which has such high bonding energy that the main skeleton thereof is not decomposed by the action of the photocatalyst and has water repellent or oil repellant organic substituent to be decomposed by action of the photocatalyst, can be listed.

Examples of such a material, which has such high bonding energy that the main skeleton thereof is not decomposed by the action of the photocatalyst and has water repellent or oil repellant organic substituent to be decomposed by action of the photocatalyst, include, for example, the materials used as the binder in “I. Case (1)”, that is, (1) the organopolysiloxanes exhibiting high strength, obtained by hydrolyzing or polycondensating chloro- or alkoxysilanes by sol-gel reaction etc. and (2) organopolysiloxanes obtained by crosslinking reactive silicone.

When such material is used as the binder in “I. Case of (1)”, the material is used as a material having cell adhesion-inhibiting properties by decomposing or denaturing the above-mentioned side chains of the organopolysiloxanes, in high ratio, so as to make it ultra-hydrophilic by the action of the photocatalyst upon irradiation with energy.

On the other hand, when such material is used as the cell adhesion-inhibiting material in this mode, the region irradiated with the energy can have cell adhesive properties by irradiating with energy to such a degree that side chains of the organopolysiloxanes are not completely decomposed or denatured by the action of the photocatalyst upon irradiation with energy.

When the above-mentioned material is used as the cell adhesion-inhibiting material, it is preferable that the material used as the cell adhesion-inhibiting material usually has a contact angle, with water, of 80° or more, particularly in the range of 100° to 130°. By this, the cell adhesive properties can be reduced. The upper limit of the angle is the upper limit of the contact angle, with water, of the cell adhesion-inhibiting material on a flat base material, and for example, when the contact angle, with water, of the cell adhesion-inhibiting material on a base material with concavoconvex is measured, the upper limit may be about 160° as shown by Ogawa et al. in Japanese Journal of Applied Physics, Part 2, Vol. 32, L614-L615, 1993.

When this cell adhesion-inhibiting material is irradiated with energy to impart the material with adhesion to cells, the material is preferably irradiated with energy such that the contact angle thereof with water comes to be in the range of 10° to 40°, particularly 15° to 30°. The cell adhesive properties can thereby be increased. The contact angle with water can be obtained by the method described above.

Together with the above-mentioned organopolysiloxane, a stable organosilicon compound not undergoing any crosslinking reaction, such as dimethylpolysiloxane, can also be separately mixed.

By using the above reactive silicone, water repellency or oil repellency can be increased, thereby decreasing interaction with cells and reducing adhesion to cells. When the above material is irradiated with energy, substituents can be easily removed to introduce OH groups etc. onto the surface, thus increasing interaction with cells to make the material excellent in cell adhesive properties.

The cell adhesion-inhibiting material is contained preferably in the range of 0.01% by weight to 95% by weight, particularly 1% by weight to 10% by weight, in the photocatalyst-containing cell adhesion-inhibiting layer. The region containing the cell adhesion-inhibiting material can thereby be a region of low cell adhesive properties.

The cell adhesion-inhibiting material preferably has surface activity. For example, when drying the photocatalyst-containing cell adhesion-inhibiting layer-forming coating solution or the like containing the cell adhesion-inhibiting material after coating thereof, the material is distributed highly unevenly on the surface of the coating film, thus giving excellent cell adhesion-inhibiting properties.

b. Others

The photocatalyst-containing cell adhesion-inhibiting layer in this mode may contain a binder and the like in accordance with required characteristics such as coating properties in formation of the layer, strength and resistance of the formed layer. The cell adhesion-inhibiting material may also function as the binder.

As the binder, for example, a binder having such high bonding energy that its main skeleton is not decomposed by the action of the photocatalyst can be used. Specific examples of the binder include polysiloxane etc. not having organic substituents or having organic substituents to such a degree that adhesion is not adversely affected, and such polysiloxane can be obtained by hydrolyzing or polycondensating tetramethoxysilane, tetraethoxysilane etc.

In this mode, the binder is contained preferably in the range of 5% by weight to 95% by weight, more preferably 40% by weight to 90% by weight, still more preferably 60% by weight to 80% by weight, in the photocatalyst-containing cell adhesion-inhibiting layer. By incorporation of the binder in this range, formation of the photocatalyst-containing cell adhesion-inhibiting layer can be facilitated and the photocatalyst-containing cell adhesion-inhibiting layer can be endowed with strength etc. thus allowing it to exhibit its characteristics.

In this mode, the photocatalyst-containing cell adhesion-inhibiting layer preferably contains a cell adhesive material having cell adhesive properties, at least after irradiation with energy. By this, the photocatalyst-containing cell adhesion-inhibiting layer can further improve the cell adhesive properties of the cell adhesive layer, that is, a cell adhesive portion as the region irradiated with energy. The cell adhesive material may be a material usable as the binder or may be a material used separately from the binder. The cell adhesive material may have excellent cell adhesive properties prior to irradiation with energy, or may be endowed with excellent cell adhesive properties by the action of the photocatalyst upon irradiation with energy. The wording “cell adhesive properties” refers to excellent adhesion to cells, and when the cell adhesive properties vary depending on the type of cell, the wording refers to excellent adhesion to the objective cells.

In this mode, as long as the cell adhesive material have excellent cell adhesive properties at least after being irradiated with energy, the cell adhesive properties can be made excellent by biological characteristics or by physical interaction such as hydrophobic interaction, electrostatic interaction, hydrogen bonding, van der Waals force.

In this mode, the cell adhesive material is contained preferably in the range of 0.01% by weight to 95% by weight, particularly 1% by weight to 10% by weight, in the photocatalyst-containing cell adhesion-inhibiting layer. By this, the photocatalyst-containing cell adhesion-inhibiting layer can further improve the cell adhesive properties of the cell adhesive layer that is a region irradiated with energy. When the material having excellent cell adhesive properties prior to irradiation with energy is used as the cell adhesive material, the material is preferably contained to such a degree as not to inhibit the cell adhesion-inhibiting properties of the cell adhesion-inhibiting material in the region not irradiated with energy, that is, the region serving as the cell adhesion auxiliary portion.

c. Method for Forming Cell Adhesive Layer

Now, the method for forming the cell adhesive layer is described in detail. In this mode as shown in FIG. 11 for example, a photocatalyst-containing cell adhesion-inhibiting layer 8 containing a photocatalyst and the cell adhesion-inhibiting material, formed on a cell culture region on a base material 1, is irradiated via a photomask 5 or the like with energy 6 in a pattern of a cell adhesive layer (cell adhesion portion) to be formed (FIG. 11A). By doing so, the cell adhesion-inhibiting material on the region irradiated with energy is decomposed or denatured thereby giving a cell adhesive layer (cell adhesion portion) 7 having cell adhesive properties, while the region not irradiated with energy can serve as a cell adhesion auxiliary portion 4 inhibiting adhesion to cells. In this case, the cell adhesion portion contains the photocatalyst and decomposed or denatured products of the cell adhesion-inhibiting material.

The energy irradiation (exposure) mentioned in this mode is a concept that includes all energy ray irradiation that can decompose or denature the cell adhesion-inhibiting material by action of a photocatalyst upon irradiation with energy, and is not limited to light irradiation.

The method for energy irradiation is the same as in the first mode in the above-mentioned “I. Case of (1)”, and thus its detailed description is omitted herein.

(2) Second Mode

Now, the mode, wherein the cell adhesion-inhibiting layer containing at least a cell adhesion-inhibiting material is formed on a photocatalyst treatment layer containing at least a photocatalyst, and the cell adhesion-inhibiting layer is irradiated with energy in a pattern of a cell adhesive layer to be formed, thereby obtaining a cell adhesion layer in which the cell adhesion-inhibiting material is decomposed and denatured, is described.

In this mode, since the cell adhesion-inhibiting layer is formed on the photocatalyst treatment layer, by irradiating the cell adhesion-inhibiting layer with energy, the photocatalyst contained in the photocatalyst treatment layer can be excited to decompose or denature the cell adhesion-inhibiting material in the cell adhesion-inhibiting layer. Thereby, a cell adhesion portion (cell adhesive layer) can be formed. In this case, the region not irradiated with energy, where the cell adhesion-inhibiting material remains, can serve as the cell adhesion auxiliary portion.

The phrase “the cell adhesion-inhibiting material is decomposed or denatured” means that the cell adhesion-inhibiting material is not contained, or that the cell adhesion-inhibiting material is contained in a smaller amount than the amount of the cell adhesion-inhibiting material contained in the cell adhesion auxiliary layer. For example, when the cell adhesion-inhibiting material is decomposed by the action of the photocatalyst upon irradiation with energy, the cell adhesion portion contains a small amount of the cell adhesion-inhibiting material, or decomposed products etc. of the cell adhesion-inhibiting material. When the cell adhesion-inhibiting material is denatured by the action of the photocatalyst upon irradiation with energy, its denatured products are contained in the cell adhesion portion. In this mode, the cell adhesion portion preferably contains the cell adhesive material having cell adhesive properties, at least after irradiation with energy. The cell adhesive properties of the cell adhesion portion can thereby be increased, and cells can adhere highly accurately only to the cell adhesion portion.

Hereinafter, the cell adhesion-inhibiting layer used in this mode is described. The photocatalyst treatment layer used in this mode can be the same as described above in the second mode in “I. Case of (1)”, and the method for forming the cell adhesive layer can be the same as in the first mode described above, and thus the description thereof is omitted herein.

(Cell Adhesion-Inhibiting Layer)

The cell adhesion-inhibiting layer used in this mode is not particularly limited insofar as it is formed on the photocatalyst treatment layer, has cell adhesion-inhibiting properties of inhibiting adhesion to cells, and contains a cell adhesion-inhibiting material to be decomposed or denatured by the action of a photocatalyst upon irradiation with energy.

In this mode, the method for forming the same is not particularly limited insofar as such layer can be formed. For example, the layer can be formed by coating a cell culture region with a cell adhesion-inhibiting layer-forming coating solution, containing the cell adhesion-inhibiting material, by a general coating method. The thickness of the cell adhesion-inhibiting layer can be suitably selected depending on the type etc. of the cell culture patterning substrate, and can usually be about 0.001 μm to 1.0 μm, particularly about 0.005 μm to 0.3 μm.

As the specific cell adhesion-inhibiting material used in the cell adhesion-inhibiting layer formed in this mode can be the same as the cell adhesion-inhibiting material used in the photocatalyst-containing cell adhesion-inhibiting layer described in the first mode, and thus its detailed description is omitted herein. Preferably, the cell adhesion-inhibiting layer in this mode contains the material having cell adhesive properties described for the photocatalyst-containing cell adhesion-inhibiting layer in the first mode. The cell adhesive properties of the cell adhesion portion (cell adhesive layer) that is a region irradiated with energy can thereby be increased.

(3) Third Mode

Now, the mode, wherein the cell adhesion-inhibiting layer containing at least a cell adhesion-inhibiting material is formed on a base material, and the cell adhesion-inhibiting layer and the photocatalyst-containing layer containing at least a photocatalyst, etc. are opposed to the cell adhesion-inhibiting layer and then irradiated with energy in a pattern of a cell adhesive layer to be formed, thereby forming a cell adhesive layer wherein the cell adhesion-inhibiting material is decomposed or denatured, is described.

In this mode, since the cell adhesion-inhibiting material decomposed or denatured by the action of a photocatalyst upon irradiation with energy is contained in the cell adhesion-inhibiting layer, the cell adhesion-inhibiting layer is arranged opposing to the photocatalyst-containing layer and then irradiated with energy in a pattern of a cell adhesive layer (cell adhesion portion) to be formed. By the action of the photocatalyst in the cell photocatalyst-containing layer, the cell adhesion-inhibiting material in the cell adhesion-inhibiting layer can be decomposed or denatured, whereby a cell adhesive layer (cell adhesion portion) can be formed. The region not irradiated with energy, wherein the cell adhesion-inhibiting material remains, can inhibit adhesion to cells and can thus be used as the cell adhesion auxiliary portion.

The phrase “the cell adhesion-inhibiting material is decomposed or denatured” means that the cell adhesion-inhibiting material is not contained, or that the cell adhesion-inhibiting material is contained in a smaller amount than the amount of the cell adhesion-inhibiting material contained in the cell adhesion auxiliary layer. For example, when the cell adhesion-inhibiting material is decomposed by the action of a photocatalyst upon irradiation with energy, the cell adhesion-inhibiting material is contained in a small amount in the cell adhesion portion (cell adhesive layer), or decomposed products etc. of the cell adhesion-inhibiting material are contained. When the cell adhesion-inhibiting material is denatured by the action of a photocatalyst upon irradiation with energy, its denatured products etc. are contained in the cell adhesion portion. In this mode, the cell adhesion portion preferably contains the cell adhesive material having cell adhesive properties, at least after irradiation with energy. The cell adhesive properties of the cell adhesion portion can thereby be increased, and cells can adhere highly accurately only to the cell adhesion portion.

The cell adhesion-inhibiting layer used in this mode is the same as the cell adhesion-inhibiting layer described above in the second mode, and the photocatalyst-containing layer side substrate and its arrangement are the same as described in the third mode in “I. Case of (1)”, and thus their detailed description is omitted herein. Further, methods, etc. for irradiating energy is the same as that of the above-described first mode, and thus their description is omitted herein.

### B. Second Embodiment

Now, the second embodiment of the cell culture patterning substrate of the present invention is described. The second embodiment of the cell culture patterning substrate of the present invention is a cell culture patterning substrate comprising: a base material; and a cell culture region which is formed on the base material, is a region for culturing a cell and contains a cell adhesive layer having adhesive properties to the cell, wherein an edge part of the cell adhesive layer is formed in a pattern with concavoconvex.

As shown in FIG. 12 for example, the cell culture patterning substrate in this embodiment is a cell culture patterning substrate having a base material 1 and a cell culture region 2 formed on the base material 1, wherein the edge part “a” of a cell adhesive layer 7 formed in the cell culture region 2 is formed in a pattern with concavoconvex.

As described above, when cells are allowed to adhere to the cell culture region, the cells are arranged regularly involving a morphological change starting from the edge part, to form a tissue. When culturing the cells, compared with a case wherein the cells are adhered along a straight line, the morphological change of the cells is more activated and can be arranged more regularly in a case wherein the cells are adhered along a line with concavoconvex. According to this embodiment, since the edge part of the cell adhesive layer, at which the arrangement of cells is initiated, is formed in a pattern with concavoconvex, the cells adhered to the edge part can be activated and the cells can be arranged highly regularly.

The following will describe each of the constituents of the cell culture patterning substrate in this embodiment. The base material used in this embodiment is the same as that used in the first embodiment described above, and thus its detailed description is omitted herein.

1. Cell Culture Region

First, the cell culture region in the cell culture patterning substrate in this embodiment is described. The cell culture region in the cell culture patterning substrate in this embodiment is not particularly limited insofar as it is a region for culturing cells and having a cell adhesive layer formed in a pattern with concavoconvex at the edge part thereof.

The cell culture region is formed on the base material, and the region of the base material other than the cell culture region serves as a non-cell culture region inhibiting adhesion to cells. The edge part of the cell adhesive layer usually serves as a boundary between this cell culture region and the non-cell culture region (shown by “a” in FIG. 12).

In this embodiment, the whole of the edge part of the cell adhesive layer formed in this cell culture region may have concavoconvex, or as shown in e.g. FIG. 12, a part of the edge part may have concavoconvex.

The concavoconvex formed in the edge part of the cell adhesive layer are preferably to an extent that the cells adhered to the cell adhesive layer can be regularly arranged. Particularly, the distance between an edge part of the concave portion and an edge part of the convex portion is preferably such a size that the cells are arranged linearly upon adhesion of the cells to the cell adhesive layer.

The specific sizes of the concavoconvex are suitably selected depending on the shape etc. of the cells to be cultured. Usually, the average distance between the edge part of the concave portion and the edge part of the convex portion of the concavoconvex is preferably in the range of 0.5 μm to 30 μm, particularly 1 μm to 5 μm. Thereby, when the cells are cultured, the cells can be cultured into an objective form without deficient cells in the edge part of the cell culture region, to form a tissue. The measurement of the average distance between the edge part of the concave portion and the edge part of the convex portion of the concavoconvex is a value determined by measuring the distances between the lowermost bottom and the uppermost top of the concavoconvex, within the range of 200 μm of the boundary between the cell adhesion portion and the cell adhesion auxiliary portion, and calculating the average thereof.

The cell adhesive layer having such edge part is not particularly limited insofar as it is a layer having cell adhesive properties. The method for forming the cell adhesive layer is not particularly limited insofar as the above-mentioned edge part can be formed. For example, the following methods can be listed: a method in which a cell adhesive layer forming coating solution etc., containing a material having cell adhesive properties, is coated by general printing methods; and a method in which, after coating the cell adhesive layer forming coating solution, the coated film is patterned by lithographic techniques etc. Moreover in this embodiment, the formation may be carried out by forming the cell adhesive layer, containing a cell adhesive material to be decomposed or denatured by the action of a photocatalyst upon irradiation with energy, and then, patterning the cell adhesive layer by irradiation with energy as described above in the first embodiment. Alternatively, the formation may be carried out by forming the cell adhesion-inhibiting layer containing a cell adhesion-inhibiting material having cell adhesive properties, and then, decomposing or denaturing the cell adhesion-inhibiting material by the action of a photocatalyst upon irradiation with energy to form a cell adhesive layer, as described above in the first embodiment.

These methods, materials etc. are the same as that in the first embodiment described above, and their detailed description is omitted herein.

In this embodiment too, the cell adhesion auxiliary portion described above in the first embodiment is preferably formed in the cell culture region described above. Cells can thereby be efficiently cultured to form a tissue, etc. having a large area.

2. Cell Culture Patterning Substrate

Now, the cell culture patterning substrate in this embodiment is described. The cell culture patterning substrate in this embodiment is not particularly limited insofar as the cell adhesive layer having the above edge part is formed on the above base material, and if necessary, members such as light-shielding portions may be formed therein.

The present invention is not limited to the above mentioned embodiments. The above mentioned embodiments are merely examples, and any one having the substantially same configuration as the technological idea disclosed in the claims of the present invention and the same effects is included in the technological scope of the present invention.

### EXAMPLES

Hereinafter, the present invention will be more specifically described by reference to the Examples.

### Example 1

(Formation of a Photomask Having a Photocatalyst-Containing Layer)

A photomask having line & space of 60 μm/300 μm with openings of 60 μm in width and light-shielding portions of 300 μm in width, wherein the boundary between the opening and the light shielding portion is formed so as to have concavoconvex of 1 by 1 μm square, was formed.

Then, 5 g of trimethoxymethylsilane TSL8114 (manufactured by GE Toshiba Silicones) and 2.5 g of 0.5 N hydrochloric acid were mixed and stirred for 8 hours. The mixture was diluted 10-fold with isopropyl alcohol to prepare a primer layer composition. This primer layer composition was coated onto the patterned surface of the photomask by spin coating, and the substrate was dried at a temperature of 150° C. for 10 minutes to form a primer layer thereon.

Then, 30 g of isopropyl alcohol, 3 g of trimethoxymethylsilane TSL8114 (manufactured by GE Toshiba Silicones), and 20 g of a photocatalyst inorganic coating agent ST-K03 (manufactured by ISHIHARA SANGYO KAISYA, LTD.) were mixed and stirred at 100° C. for 20 minutes. The mixture was diluted 3-fold with isopropyl alcohol to prepare a photocatalyst-containing layer composition. This photocatalyst-containing layer composition was coated, by spin coating, onto the photomask substrate having the primer layer formed thereon, and then dried at 150° C. for 10 minutes to form a photomask having a transparent photocatalyst-containing layer.

<Method for Forming a Cell Culture Patterning Substrate>

(Formation of a Cell Adhesion-Inhibiting Layer)

Five (5.0) grams of organosilane TSL-8114 (manufactured by GE Toshiba Silicones), 1.5 g of fluoroalkylsilane TSL-8233 (manufactured by GE Toshiba Silicones) and 2.36 g of 0.005 N hydrochloric acid were mixed and stirred for 24 hours. This solution was diluted 100-fold with isopropyl alcohol and coated by spin coating onto a quartz substrate previously subjected to alkali treatment, and the substrate was dried at a temperature of 150° C. for 10 minutes to allow hydrolysis and polycondensation reaction to advance to give a substrate having a cell adhesion-inhibiting layer of 0.2 μm in thickness.

(Patterning of the Patterning Substrate)

The cell adhesion-inhibiting layer of this substrate was opposed to the photocatalyst-containing layer of the photomask and then exposed via the photomask to ultraviolet rays with 6 J/cm² energy from a mercury lamp. Thereby, a cell culture patterning substrate having a cell adhesive surface patterned, such that the unexposed portions having cell adhesion-inhibiting properties and the exposed portions having cell adhesive properties, was obtained.

(Culture of Cells)

The cell culture patterning substrate was dipped in DMEM medium containing 10% bovine fetal serum, and primary human umbilical vein endothelial cells (HUVECs) were disseminated thereon. The cells were cultured at 37° C. in a 5% carbon dioxide atmosphere for 16 hours to allow the cells to adhere to the cell adhesion portion.

When the cells that had adhered to the cell culture substrate were observed, it was confirmed that the cells were aligned along all region in the cell culture region and were in an extended form.

Further, the DMEM medium was exchanged with one containing bFGF (Sigma) at a concentration of 10 ng/ml, culturing was continued at 37° C. in a 5% carbon dioxide atmosphere for 24 hours, and formation of a capillary tissue composed of continuous cells was confirmed.

### Comparative Example 1

The cells were cultured in the same manner as in Example 1 except that the photomask had line & space of 60 μm/300 μm without concavoconvex at the boundary between the opening and light-shielding portion. As a result, it was confirmed that the adhesion of the cells to the substrate at the time of 16 hours after cell dissemination was lower than in Example 1. After 24 hours of culturing, it was confirmed that the number of cells that had adhered to the substrate was increased, but the alignment and development of the cells were inferior to those in Example 1.

Further, shen bFGF was added to the DMEM medium similarly to Example 1 to form a tissue of the cells, the cells formed a capillary. However, it was confirmed that, as compared with Example 1, the length of the capillary was shorter and formation of the tissue was incomplete.

### Example 2

The cells were cultured in the same manner as in Example 1 except that the photomask having line & space of 190 μm/500 μm with openings of 190 μm in width and light-shielding portions of 500 μm in width, wherein 5 μm light-shielding portions were formed in every 60 μm width at the openings, was used. When the cells were observed for their shape at the time of 16 hours after cell dissemination, the alignment and development of all the cells were observed in the cell culture region.

### Comparative Example 2

The cells were cultured in the same manner as in Example 2 except that the photomask having line & space of 190 μm/500 μm was used. In this case, at the time of 24 hours after cell dissemination, the cells in the vicinity of the center part of the cell adhesion portion were adhered to the substrate but were not aligned nor extended.

### Example 3

(Formation of a Photocatalyst-Containing Cell Adhesive Layer)

3 g of isopropyl alcohol, 0.4 g of organosilane TSL8114 (manufactured by GE Toshiba Silicones), 0.04 g of N-(2-aminoethyl)-3-aminopropyltrimethoxysilane (manufactured by Huels America), and 1.5 g of a photocatalyst inorganic coating agent ST-K01 (manufactured by ISHIHARA SANGYO KAISYA, LTD.) were mixed and stirred while heating at 100° C. for 20 minutes.

This solution was coated by spin coating onto a quartz glass substrate previously subjected to alkali treatment. The substrate was dried at 150° C. for 10 minutes to allow hydrolysis and polycondensation reaction to advance, thereby forming a patterning substrate having a photocatalyst-containing cell adhesive layer, 0.2 μm in thickness, wherein the photocatalyst was strongly fixed into an organopolysiloxane, and the properties of which are changeable from cell adhesive properties to cell adhesion-inhibiting properties by the action of a photocatalyst upon irradiation with energy.

(Patterning of the Patterning Substrate)

Using a photomask having openings of 60 μm in width and light-shielding portions of 300 μm in width with line & space of 60 μm/300 μm, wherein the boundary between the opening and the light shielding portion is formed so as to have concavoconvex of 1 by 1 μm square, the patterning substrate was exposed for 900 seconds to ultraviolet rays with 300 mW/cm² intensity from a mercury lamp (wavelength 365 nm), to yield a cell culture patterning substrate having a cell adhesive surface patterned such that the unexposed portions have cell adhesive properties and the exposed portions have cell adhesion-inhibiting properties.

(Culture of Cells)

The cells were cultured in the same manner as in Example 1, and the shape of the cells was observed at the time of 16 hours after cell dissemination. It was confirmed that all the cells on the cell culture region were aligned and extended.

### Example 4

(Formation of a Photocatalyst-Containing Cell Adhesion-Inhibiting Layer)

3 g of isopropyl alcohol, 0.4 g of organosilane TSL8114 (manufactured by GE Toshiba Silicones), 0.04 g of fluoroalkylsilane TSL-8233 (manufactured by GE Toshiba Silicones), and 1.5 g of a photocatalyst inorganic coating agent ST-K01 (manufactured by ISHIHARA SANGYO KAISYA, LTD.) were mixed and stirred while heating at 100° C. for 20 minutes.

This solution was coated by spin coating onto a quartz glass substrate previously subjected to alkali treatment. The substrate was dried at 150° C. for 10 minutes to allow hydrolysis and polycondensation reaction to advance, thereby forming a patterning substrate having a photocatalyst-containing cell adhesion-inhibiting layer, 0.2 μm in thickness, wherein the photocatalyst was strongly fixed into an organopolysiloxane, and the properties of which are changeable from cell adhesion-inhibiting properties to cell adhesive properties by the action of a photocatalyst upon irradiation with energy.

(Patterning of the Patterning Substrate)

The patterning substrate was irradiated with ultraviolet rays in the same manner as in Example 3, to give a cell culture patterning substrate having a pattern wherein the unexposed portions serves as the cell adhesion-inhibiting portion and the exposed portions as the cell adhesion portion.

(Culture of Cells)

The cells were cultured in the same manner as in Example 1, and the shape of the cells was observed at the time of 16 hours after cell dissemination. It was confirmed that all the cells on the cell culture region were aligned and extended.

### Example 5

(Formation of a Photocatalyst-Containing Layer)

3 g of isopropyl alcohol, 0.4 g of organosilane TSL8114 (manufactured by GE Toshiba Silicones), and 1.5 g of a photocatalyst inorganic coating agent ST-K01 (manufactured by ISHIHARA SANGYO KAISYA, LTD.) were mixed and stirred while heating at 100° C. for 20 minutes.

This solution was coated by spin coating onto a quartz glass substrate subjected previously to alkali treatment. The substrate was dried at a temperature of 150° C. for 10 minutes to allow hydrolysis and polycondensation reaction to advance, thereby forming, on the substrate, a photocatalyst-containing layer of 0.2 μm in thickness, wherein the photocatalyst was strongly fixed into an organopolysiloxane.

(Formation of a Cell Adhesive Layer)

An aqueous solution wherein 0.2 mg of Fibronectin F-4759 (manufactured by Sigma) had been mixed with 200 ml of pure water was dropped onto the photocatalyst-containing layer, of the above-mentioned substrate provided with the photocatalyst-containing layer, at a rate of 300 μl per 1 cm² of the area of the substrate. Then, the substrate was left at 4° C. for 24 hours. Further, the substrate was cleaned with PBS for twice and was dried by exposing to a nitrogen gas to yield a patterning substrate having, on the substrate, the photocatalyst-containing layer and a cell adhesive layer.

(Patterning of the Patterning Substrate)

The patterning substrate was irradiated with ultraviolet rays in the same manner as in Example 3, to give a cell culture patterning substrate having a pattern wherein the unexposed portions serves as the cell adhesion portion and the exposed portions as the cell adhesion-inhibiting portion.

(Culture of Cells)

The cells were cultured in the same manner as in Example 1, and the shape of the cells was observed at the time of 16 hours after cell dissemination. It was confirmed that all the cells on the cell culture region were aligned and extended.

### Example 6

(Formation of a Photocatalyst-Containing Layer)

3 g of isopropyl alcohol, 0.4 g of organosilane TSL8114 (manufactured by GE Toshiba Silicones), and 1.5 g of a photocatalyst inorganic coating agent ST-K01 (manufactured by ISHIHARA SANGYO KAISYA, LTD.) were mixed and stirred under heating at 100° C. for 20 minutes.

This solution was coated by spin coating onto a quartz glass substrate subjected previously to alkali treatment. The substrate was dried at a temperature of 150° C. for 10 minutes to allow hydrolysis and polycondensation reaction to advance, thereby forming, on the substrate, a photocatalyst-containing layer of 0.2 μm in thickness, wherein the photocatalyst was strongly fixed into an organopolysiloxane.

(Formation of a Cell Adhesion-Inhibiting Layer)

This substrate was coated by spin coating with a solution comprising 5 g of isopropyl alcohol, 0.4 g of organosilane TSL8114 (manufactured by GE Toshiba Silicones) and 0.04 g of fluoroalkylsilane TSL8233 (manufactured by GE Toshiba Silicones) Then, the substrate was dried at 150° C. for 10 minutes to form a cell adhesion-inhibiting layer thereon.

(Patterning of the Patterning Substrate)

The patterning substrate was irradiated with ultraviolet rays in the same manner as in Example 3, to give a cell culture patterning substrate having a pattern wherein the unexposed portions serves as the cell adhesion-inhibiting portion and the exposed portions as the cell adhesion portion.

(Culture of Cells)

The cells were cultured in the same manner as in Example 1, and the shape of the cells was observed at the time of 16 hours after cell dissemination. It was confirmed that all the cells on the cell culture region were aligned and extended.

### Example 7

(Formation of a Cell Adhesive Layer)

3 g of isopropyl alcohol, 0.4 g of organosilane TSL8114 (manufactured by GE Toshiba Silicones) and 0.4 g of aminopropyltriethoxysilane were mixed and stirred under heating at 100° C. for 20 minutes. This solution was coated by spin coating onto a quartz glass substrate subjected previously to alkali treatment. The substrate was dried at a temperature of 150° C. for 10 minutes to allow hydrolysis and polycondensation reaction to advance, thereby forming a patterning substrate having an amino group-containing organopolysiloxane layer of about 80 nm in thickness formed on the substrate.

(Patterning of the Patterning Substrate)

The patterning substrate was irradiated with ultraviolet rays in the same manner as in Example 1, to give a cell culture patterning substrate having a pattern wherein the unexposed portions serves as the cell adhesion portion and the exposed portions as the cell adhesion-inhibiting portion.

(Culture of Cells)

The cells were cultured in the same manner as in Example 1, and the shape of the cells was observed at the time of 16 hours after cell dissemination. It was confirmed that all the cells on the cell culture region were aligned and extended.

1-6. (canceled)

7. A cell culture patterning substrate comprising:
a base material; and
a cell culture region which is formed on the base material, is a region for culturing a cell and contains a cell adhesive layer having adhesive properties to the cell,
wherein the cell culture region comprises:
a cell adhesion portion at which the cell adhesive layer is formed; and
a cell adhesion auxiliary portion, formed in a pattern, which inhibits adhesion to the cell, and
the cell adhesion auxiliary portion is formed such that, upon adhesion of the cell to the cell adhesion portion, the cells on two cell adhesion portions adjacent to the cell adhesion auxiliary portion can be bound to each other on the cell adhesion auxiliary portion.

8. The cell culture patterning substrate according to claim 7, wherein the cell adhesion auxiliary portion is formed in a line form in the cell culture region.

9. The cell culture patterning substrate according to claim 7, wherein a boundary between the cell adhesion auxiliary portion and the cell adhesion portion is formed in a pattern with concavoconvex.

10. The cell culture patterning substrate according to claim 8, wherein a boundary between the cell adhesion auxiliary portion and the cell adhesion portion is formed in a pattern with concavoconvex.

11. A cell culture patterning substrate comprising:
a base material; and
a cell culture region which is formed on the base material, is a region for culturing a cell and contains a cell adhesive layer having adhesive properties to the cell,
wherein an edge part of the cell adhesive layer is formed in a pattern with concavoconvex.

12. The cell culture patterning substrate according to claim 11, wherein the distance between an edge part of the concave portion and an edge part of the convex portion of the concavoconvex, upon adhesion of the cell to the cell adhesive layer, is a size that the cells are aligned linearly.

13. The cell culture patterning substrate according to claim 11, wherein the average distance, between the edge part of the concave portion and the edge part of the convex portion of the concavoconvex, is in the range of 0.5 μm to 30 μm.

14. The cell culture patterning substrate according to claim 12, wherein the average distance, between the edge part of the concave portion and the edge part of the convex portion of the concavoconvex, is in the range of 0.5 μm to 30 μm.
